(19) United States
(12) Patent Application Publication
(10) Pub. No.: US 2021/0040535 A1
(43) Pub. Date: Feb. 11, 2021

(54) COMPOSITIONS AND METHODS FOR SAMPLE PROCESSING

(71) Applicant: 10X GENOMICS, INC., Pleasanton, CA (US)

(72) Inventors: Adam Lowe, Pleasanton, CA (US); Christopher Hindson, Pleasanton, CA (US); Andrew D. Price, Hayward, CA (US)

(21) Appl. No.: 16/915,493

(22) Filed: Jun. 29, 2020

Related U.S. Application Data
(63) Continuation of application No. PCT/US2019/015295, filed on Jan. 25, 2019.
(60) Provisional application No. 62/622,420, filed on Jan. 26, 2018.

(51) Int. Cl. C12Q 1/6806; C12N 15/10; B01J 19/00; B01J 13/04

(52) U.S. Cl. CPC C12Q 1/6806; C12N 15/1065; B01J 2219/00547; B01J 13/046; B01J 2219/005; B01J 19/0046

(57) ABSTRACT

The present disclosure provides particles (e.g., beads) and methods, kits, and systems involving the same for sample processing or analysis. Such particles may include one or more analytes, one or more reagents, and two or more gel components and/or walled components. The particles described herein may be formed, for example, by polymerization of a polymerizable material in proximity to a gel or walled component.

### CROSS REFERENCE

This application is a continuation of International Application No. PCT/US2019/015295, filed Jan. 25, 2019, which claims the benefit of U.S. Provisional Application No. 62/622,420, filed Jan. 26, 2018, which applications are entirely incorporated herein by reference.

### BACKGROUND

Samples may be processed for various purposes, such as identification of a type of moiety within the sample. The sample may be a biological sample. The biological samples may be processed for various purposes, such as detection of a disease (e.g., cancer) or identification of a particular species. There are various approaches for processing samples, such as polymerase chain reaction (PCR) and sequencing.

Biological samples may be processed within various reaction environments, such as partitions. Partitions may be wells or droplets. Droplets or wells may be employed to process biological samples in a manner that enables the biological samples to be partitioned and processed separately. For example, such droplets may be fluidically isolated from other droplets, enabling accurate control of respective environments in the droplets.

Partitions and/or biological samples in partitions may be subjected to various processes, such as chemical processes or physical processes. Partitions and/or samples in partitions may be subjected to heating or cooling, or chemical reactions, such as to yield species that may be qualitatively or quantitatively processed.

### SUMMARY

The present disclosure provides particles for use in various sample processing and analysis applications, as well as methods, systems, and kits involving the same. The particles provided herein may include one or more analytes, one or more reagents, and two or more gel components and/or at least one gel component and at least one walled component. Such particles may be useful, for example, in controlled analysis and processing of analytes such as biological particles, nucleic acids, and proteins.

In an aspect, the present disclosure provides a particle for use in processing or analyzing an analyte from a sample, comprising: a first gel comprising a first biological material; and a second gel separate from the first gel, wherein the second gel comprises a second biological material, wherein the second gel at least partially encompasses the first gel or the first gel at least partially encompasses the second gel.

In some embodiments, the first biological material comprises the analyte. In some embodiments, the first gel further comprises a second analyte. In some embodiments, the first gel further comprises a reagent for processing or analyzing the analyte. In some embodiments, the second biological material comprises a reagent for processing or analyzing the analyte. In some embodiments, the second biological material comprises a second analyte.

In some embodiments, the second biological material comprises the analyte. In some embodiments, the second gel further comprises a second analyte. In some embodiments, the second gel further comprises a reagent for processing or analyzing the analyte. In some embodiments, the first biological material comprises a reagent for processing or analyzing the analyte.

In some embodiments, the first biological material comprises a first reagent for processing or analyzing the analyte, and the second biological material comprises a second reagent for processing or analyzing the analyte.

In some embodiments, the first gel and/or the second gel is formed by polymerization or crosslinking of polymeric precursors or macromolecules within a droplet.

In some embodiments, the second gel substantially encompasses the first gel. In other embodiments, the first gel substantially encompasses the second gel.

In some embodiments, the particle further comprises a third gel, wherein the third gel at least partially encompasses the first gel or the second gel, or is disposed between the first gel and the second gel. In some embodiments, the third gel comprises a third biological material.

In some embodiments, the first gel and the second gel are the same. In other embodiments, the first gel and the second gel are different.

In some embodiments, the analyte is a nucleic acid. In some embodiments, the analyte is a peptide.

In some embodiments, one or both of the first biological material and the second biological material comprise a reagent for processing or analyzing the analyte, wherein the reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies. In some embodiments, one or both of the first biological material and the second biological material comprise a reagent for processing or analyzing the analyte, wherein the reagent is selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

In some embodiments, the first gel and/or the second gel is disruptable or dissolvable upon application of a stimulus. In some embodiments, the first gel and the second gel are disruptable or dissolvable upon application of a stimulus. In some embodiments, the first gel and the second gel are disruptable or dissolvable upon application of the same stimulus. In some embodiments, the stimulus is selected from the group consisting of chemical triggers, bulk changes, biological triggers, light triggers, thermal triggers, magnetic triggers, and any combination thereof. In some embodiments, the stimulus is selected from the group consisting of a change in pH, a change in ion concentration, and a reducing agent. In some embodiments, the stimulus is dithiothreitol.

In some embodiments, the first gel or the second gel comprises a cell. In some embodiments, the first gel comprises a cell or cell derivative comprising the analyte.

In another aspect, the present disclosure provides a method of forming a particle for use in processing or analyzing an analyte from a sample, comprising: (a) providing a first gel comprising a first biological material; (b) generating a droplet comprising the first gel, a polymerizable material, and a second biological material; and (c) subjecting the polymerizable material to conditions sufficient to form a second gel separate from the first gel, wherein the second gel comprises the second biological material or a derivative thereof, wherein the second gel at least partially encompasses the first gel.

In some embodiments, the first biological material comprises the analyte. In some embodiments, the first gel further comprises a second analyte. In some embodiments, the first gel further comprises a reagent for processing or analyzing the analyte. In some embodiments, the second biological material or derivative thereof comprises a reagent for processing or analyzing the analyte. In some embodiments, the second biological material or derivative thereof comprises a second analyte.

In some embodiments, the second biological material or derivative thereof comprises the analyte. In some embodiments, the second gel further comprises a second analyte. In some embodiments, the second gel further comprises a reagent for processing or analyzing the analyte. In some embodiments, the first biological material comprises a reagent for processing or analyzing the analyte. In some embodiments, the first biological material comprises a first reagent for processing or analyzing the analyte, and the second biological material or derivative thereof comprises a second reagent for processing or analyzing the analyte.

In some embodiments, generating the droplet comprises flowing (i) a first phase comprising an aqueous fluid, the polymerizable material, and the second biological material and (ii) a second phase comprising a fluid that is immiscible with the aqueous fluid toward a junction, wherein, upon interaction of the first phase and the second phase, a discrete droplet of the first phase is formed.

In some embodiments, the first gel and the second gel are formed of the same polymerizable material. In other embodiments, the first gel and the second gel are formed of different polymerizable materials.

In some embodiments, the method further comprises: (d) generating a second droplet comprising the first and second gels and a second polymerizable material; and (e) subjecting the second polymerizable material to conditions sufficient to form a third gel separate from the first and second gels, wherein the third gel at least partially encompasses the second gel. In some embodiments, the method further comprises repeating (d) and (e) one or more times. In some embodiments, the third gel comprises a third biological material.

In some embodiments, the second gel encapsulates the first gel. In some embodiments, the first gel is semi-fluidic or fluidic.

In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In some embodiments, one or both of the first biological material and the second biological material or derivative thereof comprise a reagent for processing or analyzing the analyte, wherein the reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies. In some embodiments, one or both of the first biological material and the second biological material or derivative thereof comprise a reagent for processing or analyzing the analyte, wherein the reagent is selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

In some embodiments, the first gel and/or the second gel is disruptable or dissolvable upon application of a stimulus. In some embodiments, the first gel and second gel are disruptable or dissolvable upon application of a first stimulus and a second stimulus, respectively. In some embodiments, the first stimulus is the same as the second stimulus. In some embodiments, the first gel is disruptable or dissolvable upon application of the stimulus and the second gel is not disruptable or dissolvable upon application of the stimulus. In some embodiments, the second gel is disruptable or dissolvable upon application of the stimulus and the first gel is not disruptable or dissolvable upon application of the stimulus. In some embodiments, the stimulus is selected from the group consisting of chemical triggers, bulk changes, biological triggers, light triggers, thermal triggers, magnetic triggers, and any combination thereof. In some embodiments, the stimulus is selected from the group consisting of a change in pH, a change in ion concentration, and a reducing agent. In some embodiments, the stimulus is dithiothreitol.

In some embodiments, the first gel or the second gel comprises a cell. In some embodiments, the first gel or the second gel comprises a cell derivative comprising the analyte.

In another aspect, the present disclosure provides a kit comprising a plurality of particles, wherein a particle of the plurality of particles comprises (i) a first gel comprising a first biological material, and (ii) a second gel comprising a second biological material, wherein the second gel at least partially encompasses the first gel.

In some embodiments, the first gel of each particle of the plurality of particles is formed of the same polymerizable material and/or the second gel of each particle of the plurality of particles is formed of the same polymerizable material.

In some embodiments, the first biological material comprises an analyte. In some embodiments, the second biological material comprises an analyte. In some embodiments, the analyte is a cell. In some embodiments, the analyte is a nucleic acid. In some embodiments, the analyte is a protein. In some embodiments, the analyte is a lipid. In some embodiments, the analyte is selected from the group consisting of a cell, a nucleic acid, a peptide, a protein, a lipid, a transcription factor, a receptor, an antibody, and a metabolite. In some embodiments, the first biological material comprises a reagent for processing or analyzing an analyte. In some embodiments, the second biological material comprises a reagent for processing or analyzing an analyte. In some embodiments, the reagent comprises a nucleic acid barcode molecule, wherein the nucleic acid barcode molecule comprises a barcode sequence. In some embodiments, each particle of the plurality of particles comprises a nucleic acid barcode molecule, wherein the nucleic acid barcode molecule comprises a barcode sequence. In some embodiments, each particle of the plurality of particles comprises a different barcode sequence.

In another aspect, the present disclosure provides a particle for use in processing or analyzing an analyte from a sample, comprising: a first gel and a second gel separate from the first gel, wherein at least one of the first gel and the second gel comprises a biological material, and wherein the second gel at least partially encompasses the first gel.

In some embodiments, the first gel comprises the biological material and the second gel comprises an additional biological material. In some embodiments, the first gel comprises the biological material and the second gel does not include any biological material.

In some embodiments, the second gel comprises the biological material and the first gel does not include any biological material.

In some embodiments, the biological material is an analyte. In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In another aspect, the present disclosure provides a particle for use in processing or analyzing an analyte from a sample, comprising: a gel comprising a first biological material; and a walled component separate from the gel, wherein the walled component comprises a second biological material, wherein the walled component at least partially encompasses the gel or the gel at least partially encompasses the walled component.

In some embodiments, the first biological material comprises the analyte. In some embodiments, the gel further comprises a second analyte. In some embodiments, the gel further comprises a reagent for processing or analyzing the analyte. In some embodiments, the second biological material comprises a reagent for processing or analyzing the analyte. In some embodiments, the second biological material comprises a second analyte.

In some embodiments, the second biological material comprises the analyte. In some embodiments, the walled component further comprises a second analyte. In some embodiments, the walled component further comprises a reagent for processing or analyzing the analyte. In some embodiments, the first biological material comprises a reagent for processing or analyzing the analyte.

In some embodiments, the first biological material comprises a first reagent for processing or analyzing the analyte, and the second biological material comprises a second reagent for processing or analyzing the analyte.

In some embodiments, the gel or the walled component is formed by polymerization or crosslinking of polymeric precursors or macromolecules within a droplet.

In some embodiments, the walled component substantially encompasses the gel. In some embodiments, the gel substantially encompasses the walled component.

In some embodiments, the particle further comprises an additional gel, wherein the additional gel at least partially encompasses the gel or the walled component, or is disposed between the gel and the walled component. In some embodiments, the additional gel comprises a third biological material.

In some embodiments, the particle further comprises an additional walled component, wherein the additional walled component at least partially encompasses the gel or the walled component, or is disposed between the gel and the walled component. In some embodiments, the additional walled component comprises a third biological material.

In some embodiments, the gel and the walled component are formed of the same polymeric precursor material.

In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In some embodiments, one or both of the first biological material and the second biological material comprise a reagent for processing or analyzing the analyte, wherein the reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies. In some embodiments, one or both of the first biological material and the second biological material comprise a reagent for processing or analyzing the analyte, wherein the reagent is selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

In some embodiments, the gel and/or the walled component is disruptable or dissolvable upon application of a stimulus. In some embodiments, the gel and the walled component are disruptable or dissolvable upon application of a stimulus. In some embodiments, the gel and the walled component are disruptable or dissolvable upon application of the same stimulus. In some embodiments, the stimulus is selected from the group consisting of chemical triggers, bulk changes, biological triggers, light triggers, thermal triggers, magnetic triggers, and any combination thereof. In some embodiments, the stimulus is selected from the group consisting of a change in pH, a change in ion concentration, and a reducing agent. In some embodiments, the stimulus is dithiothreitol.

In some embodiments, the gel or the walled component comprises a cell. In some embodiments, the gel or the walled component comprises a cell or cell derivative comprising the analyte.

In a further aspect, the present disclosure provides a method of forming a particle for use in processing or analyzing an analyte from a sample, comprising: (a) providing a gel comprising a first biological material; (b) generating a droplet comprising the gel, a polymerizable material, and a second biological material; and (c) subjecting the polymerizable material to conditions sufficient to form a walled component separate from the gel, wherein the walled component comprises the second biological material or a derivative thereof, wherein the walled component at least partially encompasses the gel.

In some embodiments, the first biological material comprises the analyte. In some embodiments, the gel further comprises a second analyte. In some embodiments, the gel further comprises a reagent for processing or analyzing the analyte. In some embodiments, the second biological material or derivative thereof comprises a reagent for processing or analyzing the analyte. In some embodiments, the second biological material or derivative thereof comprises a second analyte.

In some embodiments, the second biological material or derivative thereof comprises the analyte. In some embodiments, the walled component further comprises a second analyte. In some embodiments, the walled component further comprises a reagent for processing or analyzing the analyte. In some embodiments, the first biological material comprises a reagent for processing or analyzing the analyte.

In some embodiments, the first biological material comprises a first reagent for processing or analyzing the analyte, and the second biological material or derivative thereof comprises a second reagent for processing or analyzing the analyte.

In some embodiments, generating the droplet comprises flowing (i) a first phase comprising an aqueous fluid, the polymerizable material, and the second biological material and (ii) a second phase comprising a fluid that is immiscible with the aqueous fluid toward a junction, wherein, upon interaction of the first phase and the second phase, a discrete droplet of the first phase is formed.

In some embodiments, the gel and the walled component are formed of the same polymerizable material. In some embodiments, the gel and the walled component are formed of different polymerizable materials.

In some embodiments, the method further comprises: (d) generating a second droplet comprising the gel and the walled component and a second polymerizable material; and (e) subjecting the second polymerizable material to conditions sufficient to form an additional gel separate from the gel and the walled components, wherein the additional gel at least partially encompasses the walled component.

In some embodiments, the method further comprises repeating (d) and (e) one or more times. In some embodiments, the additional gel comprises a third biological material.

In some embodiments, the walled component encapsulates the gel.

In some embodiments, the gel is semi-fluidic or fluidic.

In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In some embodiments, one or both of the first biological material and the second biological material or derivative thereof comprise a reagent for processing or analyzing the analyte, wherein the reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies.

In some embodiments, one or both of the first biological material and the second biological material or derivative thereof comprise a reagent for processing or analyzing the analyte, wherein the reagent is selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

In some embodiments, the gel and/or the walled component is disruptable or dissolvable upon application of a stimulus. In some embodiments, the gel and the walled component are disruptable or dissolvable upon application of a first stimulus and a second stimulus, respectively. In some embodiments, the first stimulus is the same as the second stimulus. In some embodiments, the gel is disruptable or dissolvable upon application of the stimulus and the walled component is not disruptable or dissolvable upon application of the stimulus. In some embodiments, the walled component is disruptable or dissolvable upon application of the stimulus and the gel is not disruptable or dissolvable upon application of the stimulus. In some embodiments, the stimulus is selected from the group consisting of chemical triggers, bulk changes, biological triggers, light triggers, thermal triggers, magnetic triggers, and any combination thereof. In some embodiments, the stimulus is selected from the group consisting of a change in pH, a change in ion concentration, and a reducing agent. In some embodiments, the stimulus is dithiothreitol.

In some embodiments, the gel or the walled component comprises a cell.

In another aspect, the present disclosure provides a kit comprising a plurality of particles, wherein a particle of the plurality of particles comprises (i) a gel comprising a first biological material, and (ii) a walled component comprising a second biological material, wherein the walled component at least partially encompasses the gel or the gel at least partially encompasses the walled component.

In some embodiments, the gel of each particle of the plurality of particles is formed of the same polymerizable material and/or the walled component of each particle of the plurality of particles is formed of the same polymerizable material. In some embodiments, the first biological material comprises an analyte. In some embodiments, the second biological material comprises an analyte. In some embodiments, the analyte is a cell. In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In some embodiments, the first biological material comprises a reagent for processing or analyzing an analyte. In some embodiments, the second biological material comprises a reagent for processing or analyzing an analyte. In some embodiments, the reagent comprises a nucleic acid barcode molecule, wherein the nucleic acid barcode molecule comprises a barcode sequence. In some embodiments, each particle of the plurality of particles comprises a nucleic acid barcode molecule, wherein the nucleic acid barcode molecule comprises a barcode sequence. In some embodiments, each particle of the plurality of particles comprises a different barcode sequence.

In a further aspect, the present disclosure provides a particle for use in processing or analyzing an analyte from a sample, comprising: a gel and a walled component separate from the gel, wherein at least one of the gel and the walled component comprises a biological material, and wherein the walled component at least partially encompasses the gel or the gel at least partially encompasses the walled component.

In some embodiments, the gel comprises the biological material and the walled component comprises an additional biological material. In some embodiments, the gel comprises the biological material and the walled component does not include any biological material. In some embodiments, the walled component comprises the biological material and the gel does not include any biological material.

In some embodiments, the biological material is an analyte. In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In another aspect, the present disclosure provides a particle for use in processing or analyzing an analyte from a sample, comprising: a gel comprising a first biological material; and an additional gel or a walled component separate from the gel, wherein the additional gel or the walled component comprises a second biological material, wherein the additional gel or the walled component at least partially encompasses the gel, or the gel at least partially encompasses the additional gel or the walled component.

In some embodiments, the first biological material comprises the analyte. In some embodiments, the gel further comprises a second analyte. In some embodiments, the gel further comprises a reagent for processing or analyzing the analyte. In some embodiments, the second biological material comprises a reagent for processing or analyzing the analyte. In some embodiments, the second biological material comprises a second analyte.

In some embodiments, the second biological material comprises the analyte. In some embodiments, the additional gel or the walled component further comprises a second analyte. In some embodiments, the additional gel or the walled component further comprises a reagent for processing or analyzing the analyte. In some embodiments, the first biological material comprises a reagent for processing or analyzing the analyte.

In some embodiments, the first biological material comprises a first reagent for processing or analyzing the analyte, and the second biological material comprises a second reagent for processing or analyzing the analyte.

In some embodiments, the gel or the additional gel or the walled component is formed by polymerization or crosslinking of polymeric precursors or macromolecules within a droplet.

In some embodiments, the additional gel or the walled component substantially encompasses the gel. In some embodiments, the gel substantially encompasses the additional gel or the walled component.

In some embodiments, the particle further comprises a further gel, wherein the further gel at least partially encompasses the gel or the additional gel or the walled component, or is disposed between the gel and the additional gel or the walled component. In some embodiments, the further gel comprises a third biological material.

In some embodiments, the gel and the additional gel or the walled component are the same. In some embodiments, the gel and the additional gel are the same. In some embodiments, the gel and the additional gel or the walled component are different.

In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In some embodiments, one or both of the first biological material and the second biological material comprise a reagent for processing or analyzing the analyte, wherein the reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies. In some embodiments, one or both of the first biological material and the second biological material comprise a reagent for processing or analyzing the analyte, wherein the reagent is selected from the group consisting of temperature-sensitive enzymes, pH-sensitive enzymes, light-sensitive enzymes, reverse transcriptase, proteases, ligase, polymerases, restriction enzymes, nucleases, protease inhibitors, and nuclease inhibitors.

In some embodiments, the gel and/or the additional gel or the walled component is disruptable or dissolvable upon application of a stimulus. In some embodiments, the gel and the additional gel or the walled component are disruptable or dissolvable upon application of a stimulus. In some embodiments, the gel and the additional gel or the walled component are disruptable or dissolvable upon application of the same stimulus. In some embodiments, the stimulus is selected from the group consisting of chemical triggers, bulk changes, biological triggers, light triggers, thermal triggers, magnetic triggers, and any combination thereof. In some embodiments, the stimulus is selected from the group consisting of a change in pH, a change in ion concentration, and a reducing agent. In some embodiments, the stimulus is dithiothreitol.

In some embodiments, the gel or the additional gel or the walled component comprises a cell. In some embodiments, the gel comprises a cell or cell derivative comprising the analyte.

In a further aspect, the present disclosure provides a method of forming a particle for use in processing or analyzing an analyte from a sample, comprising: (a) providing a gel comprising a first biological material; (b) generating a droplet comprising the gel, a polymerizable material, and a second biological material; and (c) subjecting the polymerizable material to conditions sufficient to an additional gel or a walled component separate from the gel, wherein the additional gel or the walled component comprises the second biological material or a derivative thereof, wherein the additional gel or the walled component at least partially encompasses the gel.

In some embodiments, the first biological material comprises the analyte. In some embodiments, the gel further comprises a second analyte. In some embodiments, the gel further comprises a reagent for processing or analyzing the analyte. In some embodiments, the second biological material or derivative thereof comprises a reagent for processing or analyzing the analyte. In some embodiments, the second biological material or derivative thereof comprises a second analyte.

In some embodiments, the second biological material or derivative thereof comprises the analyte. In some embodiments, the additional gel or the walled component further comprises a second analyte. In some embodiments, the additional gel or the walled component further comprises a reagent for processing or analyzing the analyte. In some embodiments, the first biological material comprises a reagent for processing or analyzing the analyte.

In some embodiments, the first biological material comprises a first reagent for processing or analyzing the analyte, and the second biological material or derivative thereof comprises a second reagent for processing or analyzing the analyte.

In some embodiments, generating the droplet comprises flowing (i) a first phase comprising an aqueous fluid, the polymerizable material, and the second biological material and (ii) a second phase comprising a fluid that is immiscible with the aqueous fluid toward a junction, wherein, upon interaction of the first phase and the second phase, a discrete droplet of the first phase is formed.

In some embodiments, the gel and the additional gel or the walled component are formed of the same polymerizable material. In some embodiments, the gel and the additional gel or the walled component are formed of different polymerizable materials.

In some embodiments, the method further comprises (d) generating a second droplet comprising the gel and the additional gel or the walled component and a second polymerizable material; and (e) subjecting the second polymerizable material to conditions sufficient to form a further gel separate from the gel and the additional gel or the walled component, wherein the further gel at least partially encompasses the additional gel or the walled component. In some embodiments, the method further comprises repeating (d) and (e) one or more times. In some embodiments, the further gel comprises a third biological material.

In some embodiments, the additional gel or the walled component encapsulates the gel. In some embodiments, the gel is semi-fluidic or fluidic. In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In some embodiments, one or both of the first biological material and the second biological material or derivative thereof comprise a reagent for processing or analyzing the analyte, wherein the reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies. In some embodiments, one or both of the first biological material and the second biological material or derivative thereof comprise a reagent for processing or analyzing the analyte, wherein the reagent is selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

In some embodiments, the gel and/or the additional gel or the walled component is disruptable or dissolvable upon application of a stimulus. In some embodiments, the gel and the additional gel or the walled component are disruptable or dissolvable upon application of a first stimulus and a second stimulus, respectively. In some embodiments, the first stimulus is the same as the second stimulus. In some embodiments, the gel is disruptable or dissolvable upon application of the stimulus and the additional gel or the walled component is not disruptable or dissolvable upon application of the stimulus. In some embodiments, the additional gel or the walled component is disruptable or dissolvable upon application of the stimulus and the gel is not disruptable or dissolvable upon application of the stimulus. In some embodiments, the stimulus is selected from the group consisting of chemical triggers, bulk changes, biological triggers, light triggers, thermal triggers, magnetic triggers, and any combination thereof. In some embodiments, the stimulus is selected from the group consisting of a change in pH, a change in ion concentration, and a reducing agent. In some embodiments, the stimulus is dithiothreitol.

In some embodiments, the gel or the additional gel or the walled component comprises a cell. In some embodiments, the gel or the additional gel or the walled component comprises a cell derivative comprising the analyte.

In yet another aspect, the present disclosure provides a kit comprising a plurality of particles, wherein a particle of the plurality of particles comprises (i) a gel comprising a first biological material, and (ii) an additional gel or a walled component comprising a second biological material, wherein the additional gel or the walled component at least partially encompasses the gel.

In some embodiments, the gel of each particle of the plurality of particles is formed of the same polymerizable material and/or the additional gel or the walled component of each particle of the plurality of particles is formed of the same polymerizable material. In some embodiments, the first biological material comprises an analyte. In some embodiments, the second biological material comprises an analyte. In some embodiments, the analyte is a cell. In some embodiments, the analyte is a nucleic acid. In some embodiments, the analyte is a peptide or protein.

In some embodiments, the first biological material comprises a reagent for processing or analyzing an analyte. In some embodiments, the second biological material comprises a reagent for processing or analyzing an analyte. In some embodiments, the reagent comprises a nucleic acid barcode molecule, wherein the nucleic acid barcode molecule comprises a barcode sequence. In some embodiments, each particle of the plurality of particles comprises a nucleic acid barcode molecule, wherein the nucleic acid barcode molecule comprises a barcode sequence. In some embodiments, each particle of the plurality of particles comprises a different barcode sequence.

In some embodiments, the biological material is an analyte. In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

In a further aspect, the present disclosure provides a particle for use in processing or analyzing an analyte from a sample, comprising: a gel and an additional gel or a walled component separate from the gel, wherein at least one of the gel and the additional gel or the walled component comprises a biological material, and wherein the additional gel or the walled component at least partially encompasses the gel, or the gel at least partially encompasses the additional gel or the walled component.

In some embodiments, the gel comprises the biological material and the additional gel or the walled component comprises an additional biological material. In some embodiments, the gel comprises the biological material and the additional gel or the walled component does not include any biological material. In some embodiments, the additional gel or the walled component comprises the biological material and the gel does not include any biological material.

In some embodiments, the biological material is an analyte. In some embodiments, the analyte is a nucleic acid. In some embodiments, the analyte is a peptide. In some embodiments, the analyte is a protein. In some embodiments, the analyte is a lipid. In some embodiments, the analyte is a cell. In some embodiments, the analyte is selected from the group consisting of a cell, a nucleic acid, a peptide, a protein, a lipid, a transcription factor, a receptor, an antibody, and a metabolite.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows an example of a microfluidic channel structure for partitioning individual biological particles.

FIG. 2 shows an example of a microfluidic channel structure for delivering barcode carrying beads to droplets.

FIG. 3 shows an example of a microfluidic channel structure for co-partitioning biological particles and reagents.

FIG. 4 shows an example of a microfluidic channel structure for the controlled partitioning of beads into discrete droplets.

FIG. 5 shows an example of a microfluidic channel structure for increased droplet generation throughput.

FIG. 6 shows another example of a microfluidic channel structure for increased droplet generation throughput.

FIG. 7 shows an example of a particle including two gel components or gels.

FIG. 8 shows an example of a particle including multiple gel components or gels.

FIG. 9 shows a computer system that is programmed or otherwise configured to implement methods provided herein.

FIG. 10 shows an example of a particle including a gel and a walled component.

FIG. 11 shows an example of a particle including a gel and a walled component.

FIG. 12 shows an example of a particle including multiple gels and walled components.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

The term “barcode,” as used herein, generally refers to a label, or identifier, that conveys or is capable of conveying information about an analyte or partition. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing-reads.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. The subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient.

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “sample,” as used herein, generally refers to a biological sample of a subject. The biological sample may comprise any number of macromolecules, for example, cellular macromolecules. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

The term “biological particle,” as used herein, generally refers to a discrete biological system derived from a biological sample. The biological particle may be a virus. The biological particle may be a cell or derivative of a cell. The biological particle may be an organelle. The biological particle may be a rare cell from a population of cells. The biological particle may be any type of cell, including without limitation prokaryotic cells, eukaryotic cells, bacterial, fungal, plant, mammalian, or other animal cell type, mycoplasmas, normal tissue cells, tumor cells, or any other cell type, whether derived from single cell or multicellular organisms. The biological particle may be or may include a matrix (e.g., a gel or polymer matrix) comprising a cell or one or more constituents from a cell (e.g., cell bead), such as DNA, RNA, organelles, proteins, or any combination thereof, from the cell. The biological particle may be obtained from a tissue of a subject. The biological particle may be a hardened cell. Such hardened cell may or may not include a cell wall or cell membrane. The biological particle may include one or more constituents of a cell, but may not include other constituents of the cell. An example of such constituents is a nucleus or an organelle. A cell may be a live cell. The live cell may be capable of being cultured, for example, being cultured when enclosed in a gel or polymer matrix, or cultured when comprising a gel or polymer matrix.

The term “macromolecular constituent,” as used herein, generally refers to a macromolecule contained within or from a biological particle. The macromolecular constituent may comprise a nucleic acid. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may small RNA that are less than 200 nucleic acid bases in length, or large RNA that are greater than 200 nucleic acid bases in length. Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. The partition may isolate space or volume from another space or volume. The partition may be a droplet or well, for example. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase.

Provided herein are particles (e.g., beads) that may be used for various purposes, such as in kits, methods, or systems for sample processing or analysis. Such particles may include one or more biological materials (e.g., one or more analytes or reagents) and two or more gels (e.g., gel components). One gel (e.g., a gel component) may include a first biological material (e.g., an analyte) and another gel may include a second biological material (e.g., a reagent for processing or analyzing an analyte). Alternatively, one gel may include a biological material and the second gel may not include any biological material. A reagent for processing or analyzing an analyte may be provided to the analyte upon, for example, disruption or dissolution of a gel. Processing or analysis of the analyte may be carried out within a partition, e.g., a droplet or a well. The particles disclosed herein may be formed by polymerizing or cross-linking a polymerizable material or macromolecule around or in proximity to a gel including a biological material such as an analyte or a reagent.

### Multi-Component Particles

In an aspect, the present disclosure provides a particle for use in processing or analyzing an analyte from a sample. The particle may comprise a first gel comprising a first biological material (e.g., the analyte) and a second gel. The second gel may be separate from the first gel. For example, the second gel and the first gel may be comprised of different materials, have been prepared according to different processes and/or at different times, and/or be physically separated from one another (e.g., with the first gel encompassing the second gel as a distinct layer, or vice versa). For example the first gel may be distinct from the second gel. The second gel may comprise a second biological material (e.g., another analyte or a reagent for processing or analyzing an analyte) or may not include any biological material. The second gel may at least partially encompass the first gel. Alternatively, the first gel may at least partially encompass the second gel. The first gel may be disruptable or dissolvable upon application of a stimulus. In some cases, the second gel may be disruptable or dissolvable upon application of a stimulus.

The particle may comprise multiple layers. In some cases, the first gel is a first layer and the second gel is or approximates a second layer of the particle. The first layer may be partially or completely encompassed or surrounded by the second layer, or vice versa. For example, the first layer may be a center (e.g., core) of the particle and the second layer may be a coating on the first layer. In this instance, the second and first layers may be substantially concentric. In other cases, the second gel may encapsulate at least about 5%, 10%, 15%, 20%, 25%, 30%, 35%, 40%, 45%, 50%, 55%, 60%, 65%, 70%, 75%, 80%, 85%, 90%, 95%, 97%, 98%, or 99% of the first gel. Similarly, the second layer comprising the second gel may be a center (e.g., core) of the particle and the first layer comprising the first gel may be a coating on the second layer. The first gel may encapsulate at least about 5%, 10%, 15%, 20%, 25%, 30%, 35%, 40%, 45%, 50%, 55%, 60%, 65%, 70%, 75%, 80%, 85%, 90%, 95%, 97%, 98%, or 99% of the second gel. A particle core may comprise a semi-fluidic (e.g., mixture of solid and liquid, or semi-solid) or fluidic material. For example, the particle may comprise a gel layer encapsulating a liquid core, a fluid or semi-fluid layer surrounding a solid core. The fluid or semi-fluid layer may be surrounded by one or more other layers, such as a solid or semi-solid layer.

The first gel and/or the second gel may be formed by polymerization of polymeric precursors within a droplet, as described elsewhere herein. For example, the first gel may be formed by providing polymeric precursors (e.g., monomers) within a droplet and subjecting the droplet to a stimulus (e.g., ultraviolet light) to induce polymerization or crosslinking. The second gel may be similarly formed. In some cases, the second gel may be formed by providing polymeric precursors within a partition (e.g., a droplet or well) including the first gel comprising a first biological material (e.g., an analyte or a reagent) and subjecting the partition to a stimulus to induce polymerization. The second gel may form around or adjacent to the first gel, as described herein. The second gel may comprise a second biological material (e.g., an analyte or reagent). In other cases, the first gel comprising a first biological material (e.g., an analyte or a reagent) may be formed by polymerizing polymeric precursors within a droplet including the second gel such that the first gel forms around or adjacent to the second gel. The second gel may comprise a second biological material (e.g., an analyte or reagent). Particles including one or more gel components may be formed according to the droplet generation methods described elsewhere herein. During formation of a gel, a biological material may undergo a chemical or physical change. For example, a biological material may undergo crosslinking with a polymerizable material used to form a gel. Accordingly, a gel may include a derivative of a biological material.

The particle may include at least 2, 3, 4, 5, 6, 7, 8, 9, 10, 15, 20, 25, 30, 35, 40, 45, 50, 55 60, 65, 70, 75, 80, 85, 90, 95, 100, or more gels. One or more gels included in a multi-gel particle may comprise the same material (e.g., be formed of the same polymeric precursors). Gels comprising the same material may be separately formed and/or separately disposed within a particle. For example, a particle may include two gels (e.g., gel components) comprising the same material (e.g., a first layer including an analyte and a second layer formed of the same material and including one or more reagents). Alternatively, a particle may include two or more gels comprising different materials. In some cases, a particle may include alternating gel layers comprising two or more materials. For example, a particle may include a first layer comprised of a first material, a second layer comprised of a second material that is different from the first material, a third layer comprised of a third material that is the same as the first material, a fourth layer comprised of a fourth material that is the same as the second material, etc. Alternatively, each gel of a multi-gel particle may comprise a different material. Such gels may be situated in separate or discrete layers. The polymeric precursors used to form each gel may be as described elsewhere herein.

For a particle including multiple discrete layers of gels, each layer may have the same or a different thickness. For example, a particle may include a first inner layer (e.g., a core), a second layer surrounding the first layer, and a third layer surrounding the second layer. The second and third layers may have the same or different thickness. Similarly, each gel (e.g., gel component) of the particle may have the same or different surface area. For particles including multiple discrete layers, the surface area of an inner layer (e.g., the core of the particle) may be smaller than the surface area of an outer layer (e.g., a first layer coating the core of the particle). Each gel of the particle may also have the same or different volumes. For example, the first gel may have a larger volume (e.g., 1%, 5%, 10%, 15%, 20%, 30%, 50%, 75%, 100%, or a greater amount larger) than the second gel. Alternatively, the second gel may have a larger volume (e.g., 1%, 5%, 10%, 15%, 20%, 30%, 50%, 75%, 100%, or a greater amount larger) than the first gel. Differences in thickness, surface area, and volume of different gel components of a particle may result from, for example, different amounts or types of polymeric precursors used to form each gel, characteristics of the gel (e.g., water content, density, or tightness of packing), or contents of the gel (e.g., size and/or concentration of an analyte or reagent included therein).

One of more gel components of a particle may be substantially porous or substantially non-porous. A gel component may be substantially solid, semi-solid, semi-fluidic, or fluidic. For example, a particle may include a first gel layer that is fluidic or semi-fluidic surrounded by a second gel layer that is semi-solid. One or more gel components of a particle may be rigid. Similarly, one or more gel components may be flexible and/or compressible.

Properties of a gel or combination of gels in a particle may be tailored based on a desired property or feature of a particle. For example, in cases in which a first gel encompasses a second gel and does not include a biological material, properties of the first gel may be tailored to regulate a rate at which the biological material in the second gel becomes accessible. For example, a thickness of the first gel may be selected such that a rate of dissolution of the first gel impacts a time period within which disruption or dissolution of the second gel is initiated.

A particle comprising multiple gel components may have any useful shape and size. For example, a particle may be spherical or substantially spherical, e.g., in the instance of a particle having multiple concentric layers. Alternatively, a particle shape may be ovular, oblong, circular (e.g., disc-like), cylindrical, or amorphous. In one example, a particle may have a dumbbell shape. Such a particle may have a first gel component comprising a first cell and a second gel component comprising a second cell, where the first and second gel components meet or overlap between the two cells. A particle may have a dimension (e.g., a diameter) that is at least about 1 nanometer (nm), 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. Alternatively, a particle may have a dimension (e.g., a diameter) that is less than about 100 nm, 500 nm, 5 μm, 10 μm, 20 μm, 30 μm, 40 μm, 50 μm, 60 μm, 70 μm, 80 μm, 90 μm, 100 μm, 250 μm, 500 μm, or 1 mm, or less. In the case of a particle having a dumbbell shape, the diameter of the first gel component may be the same or different from the diameter of the second gel component. For example, the diameter of the first gel component may be smaller than the diameter of the second gel component.

A particle comprising multiple gel components may be used to analyze an analyte (e.g., an analyte of interest) using any useful reagent or combination of reagents, including but not limited to those described elsewhere herein. A multi-component particle may include a biological particle (e.g., a cell) or a component thereof, such as a nucleic acid. An analyte may also be, for example, a peptide, a protein, a lipid, a transcription factor, a receptor, an antibody, or a metabolite. Reagents for analyzing an analyte of interest may be selected from the non-limiting group consisting of enzymes, fluorophores, oligonucleotides, primers, barcodes, nucleic acid barcode molecules (e.g., nucleic acid barcode molecules comprising one or more barcode sequences), buffers, deoxynucleotide triphosphates, detergents, reducing agents, chelating agents, oxidizing agents, nanoparticles, and antibodies. In some cases, one or more reagents is selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

An analyte may be disposed in any useful location within a particle. For example, the analyte may be contained within a centrally located gel (e.g., a first gel) surrounded by an outer layer (e.g., a second gel) containing one or more reagents. Alternatively, an analyte may be contained within a gel layer (e.g., a first gel) that overlays a gel (e.g., a second gel) containing one or more reagents. Particles may also include more than one analyte for analyzing and processing. For example, the first gel may include multiple analytes. Alternatively, the second gel or another gel (e.g., a third gel, a fourth gel, or any other gel) may include an analyte in addition to a first analyte included in the first gel. This second analyte may be the same or different from the first analyte. For example, a particle may include two or more analytes selected from the non-limiting group consisting of biological particles (e.g., cells or cell beads), nucleic acids, proteins, peptides, lipids, transcription factors, receptors, antibodies, and metabolites. In some cases, a particle may include multiple cells.

Similarly, one or more reagents may be disposed in any useful location within a particle. For example, the first gel may include an analyte and a first reagent, and the second gel may at least partially encompass the first gel and include a second reagent. The first and second reagents may be the same or different from one another. In one example, the first gel comprises an analyte that is a cell and a first reagent capable of lysing the cell to release a component of the cell, while the second gel comprises a second reagent useful for analyzing or processing the component of the cell. In some cases, the first reagent and the cell may be separately disposed or fixated within the first gel such that lysing of the cell does not occur. Application of an appropriate stimulus to disrupt or dissolve the first gel (e.g., as described herein) may permit the first reagent to come into contact with the cell to release the component of the cell. The cellular component may then be available to the second reagent of the second gel for analyzing or processing. In some cases, the second gel must be disrupted or dissolved (e.g., by application of an appropriate stimulus) to make the second reagent available to the cellular component.

One or more gels of a particle may be disrupted or dissolved by application of a stimulus. For example, a first gel may be disruptable or dissolvable by application of a stimulus and a second gel may not be disruptable or dissolvable by application of a stimulus, or vice versa. Application of a stimulus may disrupt or dissolve one or more gels of a particle. In some cases, multiple gels of a particle may be disrupted or dissolved by the same stimulus simultaneously or sequentially (e.g., one after another). For example, a stimulus may disrupt or dissolve a second gel that substantially encompasses a first gel and subsequently disrupt or dissolve the first gel. Such a stimulus may be, for example, a chemical agent requiring a single application (e.g., introduction). The chemical agent may first interact with, and consequently disrupt or dissolve, the second gel, and then, subsequent to the disruption or dissolution of the second gel, interact with the first gel. In another example, such a stimulus may be a photo-stimulus or thermal stimulus that requires multiple applications to disrupt or dissolve both gels. The first application of a photo-stimulus or thermal stimulus may disrupt or dissolve all or a portion of the second gel, and a subsequent application of the photo-stimulus or thermal stimulus may disrupt or dissolve all or a portion of the first gel. Alternatively, a separate stimulus may be necessary for the disruption or dissolution of each gel. A stimulus may be selected from the non-limiting group consisting of chemical triggers, bulk changes, biological triggers, light triggers, thermal triggers, magnetic triggers, and any combination thereof. A stimulus may be a change in pH, a change in ion concentration, or a reducing agent. For example, a stimulus useful for disrupting or dissolving a gel component of a multi-component particle may be dithiothreitol. In one example, the first gel comprises an analyte and is surrounded by the second gel comprising a reagent, and the first gel is capable of disruption or dissolution by changing pH or an ion concentration. In another example, the first gel comprises an analyte and is surrounded by the second gel comprising a reagent, and the second gel is capable of disruption or dissolution by changing pH or an ion concentration. In yet another example, the first gel comprises an analyte and is surrounded by the second gel comprising a reagent, and the second gel is capable of disruption or dissolution by exposure to dithiothreitol.

In some examples, a stimulus is a chemical or biological stimulus included in the particle (e.g., a reducing agent in an outer layer of the particle). In such a case, the outer layer may be disrupted upon application of another stimulus (e.g., light) to release the chemical or biological stimulus, for example.

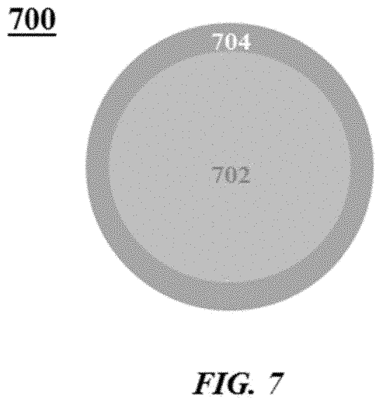

FIG. 7 shows a cross-section of a particle 700 including gel components 702 and 704 in discrete layers. Gel components 702 and 704 may be formed of the same or different materials and may be substantially concentric. Gel component 702 may include a biological material such as an analyte and/or a reagent. Gel component 702 may be semi-fluidic or fluidic. Gel component 704 may also include a biological material such as an analyte and/or a reagent. In one example, gel component 702 comprises a cell and gel component 704 comprises one or more reagents selected from the group consisting of nucleic acid barcode molecules, oligonucleotides, barcodes, primers, polymerases, dNTPs, co-factors, and buffers. The analyte and the one or more reagents may be fixated within their respective gel components such that they do not come into contact with one another. Disruption or dissolution of gel component 704 may provide one or more reagents to the cell for processing and analysis. Alternatively, disruption or dissolution of gel component 702 may facilitate contact between the cell and the one or more reagents. Disruption or dissolution of gel component 702 may also result in lysis of the cell included therein such that one or more components of the cell are released. In some cases, gel components 702 and 704 are disrupted or dissolved simultaneously or successively. Upon disruption or dissolution of both gel components, the cell (or, in the instance of a lysed cell, the components thereof) and the one or more reagents may be disposed within a partition such as a droplet. The cell or its components may come into contact with the one or more reagents for processing. For example, a nucleic acid included within the cell prior to its lysing may be exposed to one or more reagents useful for nucleic acid amplification. Nucleic acid amplification may then be carried out, e.g., as described elsewhere herein.

In another example, gel component 702 includes an mRNA and gel component 704 includes one or more reagents selected from the group consisting of reverse transcriptase, primers, and template switching oligos. As in the previous example, dissolution or disruption of one or both of gel components 702 and 704 may provide for contact between the mRNA analyte and the one or more reagents to facilitate, e.g., generation of cDNA, as described elsewhere herein.

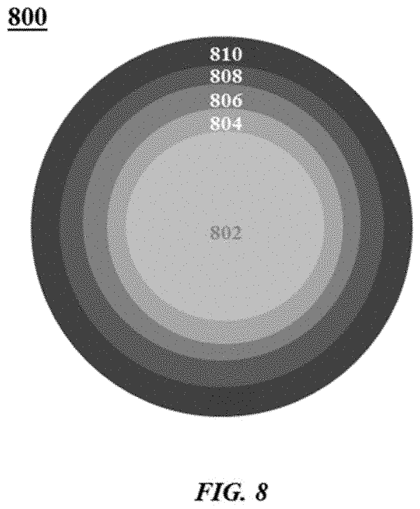

FIG. 8 shows a cross-section of a particle 800 including gel components 802, 804, 806, 808, and 810 in discrete layers. Each gel component may be formed of a different material, or two or more gel components may be formed of the same material. For example, gel components 802, 806, and 810 may be formed from the same polymeric precursors while gel components 804 and 808 may be formed of the different polymeric precursors. One or more of gel components 802, 804, 806, and 808 may be semi-fluidic or fluidic. Each gel component may include a biological material such as an analyte or a reagent. Two or more of the gel components may include an analyte of interest. For example, every other layer (e.g., gel components 802, 806, and 810 or gel components 804 and 808) of particle 800 may include an analyte. Analytes included in different gel components may be the same or different. For example, gel component 804 may include a cell while gel component 808 may include a protein. Similarly, two or more of the gel components may include a reagent for processing or analyzing an analyte. A single gel component may include one or more analytes and one or more reagents.

In another aspect, the present disclosure provides a method of forming a particle for use in processing or analyzing an analyte from a sample, comprising providing a first gel comprising a first biological material; generating a partition (e.g., a droplet or well) comprising the first gel, a polymerizable material, and a second biological material; and subjecting the polymerizable material to conditions sufficient to form a second gel. The second gel may be separate from the first gel. For example, the second gel and the first gel may be comprised of different materials, have been prepared according to different processes and/or at different times, and/or be physically separated from one another (e.g., with the first gel encompassing the second gel as a distinct layer, or vice versa). For example the first gel may be distinct from the second gel. In some cases, the second gel at least partially encompasses the first gel. The first biological material may comprise the analyte or a reagent for processing or analyzing the analyte. Similarly, the second biological material may comprise the analyte or a reagent for processing or analyzing the analyte. At least one of the first gel and the second gel may comprise the analyte. Alternatively, neither the first gel nor the second gel may comprise the analyte. At least one of the first gel and the second gel may comprise a reagent for processing or analyzing the analyte. For example, the first gel may comprise the analyte while the second gel comprises one or more reagents for processing or analyzing the analyte. In another example, the first gel comprises the analyte and at least one reagent while the second gel comprises another reagent that is the same or different from a reagent in the first gel. In a further example, the first gel and the second gel each comprise a reagent for processing or analyzing an analyte.

The method of forming a particle comprising multiple gel components may be used to form any multi-component particle described herein.

The first gel of the particle may be formed as described herein (e.g., using microfluidics methods, air knife droplet generation, aerosol generation, or a membrane based encapsulation system). Generating the partition (e.g., droplet) comprising the first gel, a polymerizable material, and a biological material (e.g., an analyte and/or one or more reagents for processing or analyzing an analyte) may comprise flowing (i) a first phase comprising an aqueous fluid, the polymerizable material, and the biological material and (ii) a second phase comprising a fluid that is immiscible with the aqueous fluid toward a junction. Upon interaction of the first and second phases, a discrete droplet of the first phase may be formed. The polymerizable material may then be subjected to a stimulus capable of polymerizing it into a gel. The stimulus may be selected from, for example, thermal stimuli (e.g., heating or cooling), photo-stimuli (e.g., through photo-curing), chemical stimuli (e.g., through crosslinking or added initiators), and any combination thereof. The stimulus capable of polymerizing the polymerizable material into a gel may be a material included in the first phase. Such a stimulus may be capable of polymerizing the polymerizable material into a gel in situ. The polymerizing may comprise subunit addition and/or cross-linking.

The particle generation method described herein may further comprise generating a second droplet comprising the first and second gels and a second polymerizable material and subjecting the second polymerizable material to conditions sufficient to form a third gel separate from the first and second gels. The third gel may at least partially encompass the second gel. These steps may be repeated one or more times to form a particle including three or more (e.g., four, five, six, seven, eight, nine, ten, or more) gel components. The third gel and any other additional gel may include a biological material (e.g., an analyte or a reagent for processing or analyzing an analyte).

In some cases, the first and second gels may be formed of the same polymerizable material, as described herein. In other cases, the first and second gels may be formed of different polymerizable materials. In one example, a particle may include a first gel and a third gel formed of the same polymerizable material and a second gel formed of a different polymerizable material, e.g., in an alternating pattern.

A particle formed by the disclosed method may include one or more layers, as described herein. For example, a particle may include a first layer (e.g., a core) comprising a first gel and a second layer comprising a second gel. A core may comprise a semi-fluidic or fluidic material. The core may comprise a semi-solid material. For example, the particle may comprise a gel layer encapsulating a liquid or semi-liquid core. The particle may include one or more additional layers overlaying the second layer. The second layer may partially or completely encompass the first layer.

An analyte may be included in either the first gel or the second gel, as described herein. In some cases, multiple gel components of a particle may include an analyte. For example, both the first gel and the second gel may include an analyte. These analytes may be the same or different. Analytes may be, for example, biological particles (e.g., cells) or components thereof, nucleic acids, peptides, proteins, lipids, transcription factors, receptors, antibodies, metabolites, or any other analyte of interest. In certain cases, one or more gel components of a particle may include a cell.

At least one of the first gel and the second gel may include one or more reagents, as described herein. The first gel may comprise an analyte and one or more reagents may be included in the second gel. Alternatively, the first gel may comprise one or more reagents and the second gel may comprise an analyte. In other cases, both the first and second gels may include analytes, or the first and second gels may include reagents. Particles may include one or more reagents in multiple gel components. For example, a particle may have two gel components that each include at least one reagent. Reagents included in different gel components of a particle may be the same or different. A reagent included in a particle made by a method of the present disclosure may be any useful reagent to achieve any useful purpose toward analyzing and processing an analyte. One or more reagents may be selected from the non-limiting group consisting of enzymes, fluorophores, oligonucleotides, primers, barcodes, buffers, deoxynucleotide triphosphates, detergents, reducing agents, chelating agents, oxidizing agents, nanoparticles, and antibodies. One or more reagents may also be selected from the non-limiting group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

At least one gel of a particle formed by the methods disclosed herein may be disruptable or dissolvable upon application of a stimulus, as described herein. In some cases, the first gel may be disruptable or dissolvable upon application of a stimulus. In other cases, the second gel may be disruptable or dissolvable upon application of a stimulus. A stimulus capable of disrupting or dissolving one or more gel components of a particle may be selected from the non-limiting group consisting of chemical triggers, bulk changes, biological triggers, light triggers, thermal triggers, magnetic triggers, and any combination thereof. In some instances, a stimulus may be selected from the non-limiting group consisting of a change in pH, a change in ion concentration, and a reducing agent such as dithiothreitol.

In another aspect, the present disclosure provides a kit including a plurality of particles each comprising one or more gels. For example, a kit may include an array of particles (e.g., 2, 4, 6, 8, 10, 20, 40, 60, 80, 100, 200, 300, 400, 500, 1000, or more particles). The particles included in a kit may be the same (e.g., comprising the same gels, analytes, reagents, and configuration) or different. A kit may comprise a plurality of particles, in which a particle of the plurality of particles comprises (i) a first gel including a first biological material (e.g., an analyte or a reagent) and (ii) a second gel comprising a second biological material (e.g., an analyte or a reagent). The first gel may be separate from the second gel. For example, the second gel and the first gel may be comprised of different materials, have been prepared according to different processes and/or at different times, and/or be physically separated from one another (e.g., with the first gel encompassing the second gel as a distinct layer, or vice versa). For example the first gel may be distinct from the second gel. The second gel may at least partially encompass the first gel. The first gel of each particle of the plurality of particles may be formed of the same polymerizable material. The second gel of each particle of the plurality of particles may also or alternatively be formed of the same polymerizable material. The polymerizable material of the first gel may be the same or different from the polymerizable material of the second gel. The first biological material and/or the second biological material may comprise an analyte, such as a cell or nucleic acid. The particles may each include a cell. The cells may derive from the same organism or a component thereof (e.g., a tissue) or the same cell line or may derive from different sources. Alternatively, the particles may each include a nucleic acid that is the same or different in each particle.

The first biological material and/or the second biological material may also or alternatively comprise a reagent for processing or analyzing an analyte. A particle may include one or more reagents disposed in the same or another gel. A reagent may comprise a nucleic acid barcode molecule that may include a barcode sequence. In one example, each particle of the plurality of particles may comprise a nucleic acid barcode molecule including a barcode sequence. Each particle of the plurality of particles may include a different barcode sequence. A particle of the plurality of particles may include a plurality of nucleic acid barcode molecules, where each of the plurality of nucleic acid barcode molecules includes a barcode sequence, where the barcode sequence is the same for each nucleic acid barcode molecules of the plurality of nucleic acid barcode molecules. Each nucleic acid barcode molecule of the plurality of nucleic acid barcode molecules may also include a second barcode sequence. This second barcode sequence may vary between the nucleic acid barcode molecules of the plurality of nucleic acid barcode molecules associated with a given particle. For example, all or a subset of the plurality of nucleic acid barcode molecules associated with a given particle may have a different second barcode sequence.

In another aspect, the present disclosure provides a kit including a plurality of particles each comprising a single gel and a polymerizable material. Such a kit may include, for example, an array of particles (e.g., 2, 4, 6, 8, 10, 20, 40, 60, 80, 100, 200, 300, 400, 500, 1000, or more particles) each including a single gel. The single gel of each particle of the plurality of particles may be comprised of the same polymerizable material. The particles of the plurality of particles may include a biological material such as an analyte or a reagent for processing or analyzing an analyte. The analyte may be a cell, a nucleic acid, protein, lipid, transcription factor, metabolite, antibody, or a peptide, as described herein. The reagent may comprise a nucleic acid barcode molecule that may include a barcode sequence. In one example, each particle of the plurality of particles may comprise a nucleic acid barcode molecule including a barcode sequence. Each particle of the plurality of particles may include a different barcode sequence. A particle of the plurality of particles may include a plurality of nucleic acid barcode molecules, where each of the plurality of nucleic acid barcode molecules includes a barcode sequence, where the barcode sequence is the same for each nucleic acid barcode molecules of the plurality of nucleic acid barcode molecules. Each nucleic acid barcode molecule of the plurality of nucleic acid barcode molecules may also include a second barcode sequence. This second barcode sequence may vary between the nucleic acid barcode molecules of the plurality of nucleic acid barcode molecules associated with a given particle. For example, all or a subset of the plurality of nucleic acid barcode molecules associated with a given particle may have a different second barcode sequence. The particles may also include one or more additional reagents.

The polymerizable material provided in the kit may be the same as or different from that used to form the first gel of the particles. The kit may be provided with one or more cells or nucleic acids, or with one or more reagents. Such a kit may be used according to the methods disclosed herein. For example, a particle of the kit comprising a single gel comprising a nucleic acid barcode molecule including a barcode sequence may be combined in a partition (e.g., a droplet) with a polymerizable material and an analyte of interest (e.g., a cell or nucleic acid that may or may not be included with the kit). The polymerizable material may then be subjected to conditions sufficient to form a second gel separate from the first gel that includes the analyte of interest. The particle created thereby may be used, e.g., as described elsewhere herein.

In a further aspect, the present disclosure provides a particle for use in processing or analyzing an analyte from a sample, which particle comprises a gel and a walled component. The walled component may be separate from the gel. For example, the gel and the walled component may be comprised of different materials, have been prepared according to different processes and/or at different times, and/or be physically separated from one another (e.g., with the gel encompassing the walled component as a distinct layer, or vice versa). For example the gel may be distinct from the walled component. The gel may comprise a first biological material (e.g., the analyte) and the walled component may comprise a second biological material (e.g., another analyte or a reagent for processing or analyzing an analyte) or may not include any biological material. Alternatively, the walled component may comprise a first biological material (e.g., the analyte) and the gel may comprise a second biological material (e.g., another analyte or a reagent for processing or analyzing an analyte) or may not include any biological material. The walled component may at least partially encompass the gel. Alternatively, the gel may at least partially encompass the walled component. The gel may be disruptable or dissolvable upon application of a stimulus. The walled component may be disruptable or dissolvable upon application of a stimulus. A stimulus capable of disrupting or dissolving a gel may be different than a stimulus capable of disrupting or dissolving a walled component.

A walled component may comprise a layer that partially or completely surrounds one or more other components. For example, the layer (e.g., wall) of a walled component may partially or completely surround a fluid or semi-fluid material, such as an aqueous solution. The one or more components disposed within a walled component may be retained by the walled component alone or by the walled component in combination with one or more other features. For example, the walled component may partially encapsulate the one or more components and a separate gel layer may complete the encapsulation such that the walled component and the gel layer together encapsulate the one or more components. The one or more components retained partially or completely by a walled component may comprise, for example, an aqueous solution comprising one or more analytes (e.g., one or more cells, cell beads, peptides, proteins, lipids, transcription factors, receptors, metabolites, or nucleic acid molecules, as described herein) and/or one or more reagents for processing analytes (e.g., as described herein). For example, a walled component may encapsulate an aqueous solution comprising one or more reagents. In another example, a walled component may encapsulate an aqueous solution comprising one or more analytes.

The walled component may define any shape of any dimensions. The shape and dimensions of the walled component may be dependent upon the one or more components included therein and/or other components disposed outside of the walled component. A wall of a walled component may be permeable or semi-permeable. Alternatively, a wall may be impermeable. A wall may be solid, rigid, semi-solid, or fluidic. In some cases, a wall may be or comprise a membrane, such as a lipid layer or lipid bilayer. A wall may comprise a gel or polymeric material (e.g., as described herein). Alternatively, a wall may not comprise a gel. For example, a wall may comprise a lipid wall such as a lipid membrane. A wall may be degradable or dissolvable, such as upon application of a stimulus (e.g., as described herein). For example, a wall of a walled component may comprise a degradable polymeric material that is degradable upon application of a stimulus, such as a chemical or thermal stimulus. A wall of a walled component may be single or multi-layered. For example, a wall may comprise a first layer formed of a first polymeric material and a second layer formed of a second polymeric material. The first polymeric material may differ from the second polymeric material and have one or more different properties, such as different thicknesses and resistances or susceptibilities to certain stimuli. A walled component having multiple walls having different properties may facilitate controlled degradation or dissolution of the walls to provide access to the one or more components therein. In an example, a first wall layer may dissolve upon application of a first stimulus and a second wall layer disposed interior to the first wall layer may dissolve upon application of a second stimulus that is different from the first stimulus. A given wall (e.g., the sole wall or a wall of multiple walls) of a walled component may have any desired thickness and may include one or more components embedded therein or attached thereto. One or more components may be attached to an interior or an exterior of the wall. For example, a wall of a walled component may comprise a plurality of oligonucleotides (e.g., a plurality of nucleic acid barcode molecules) coupled thereto (e.g., on an interior or exterior of the wall). In another example, a wall of a walled component may comprise a plurality of analytes and/or a plurality of reagents for use in analyzing one or more analytes coupled thereto (e.g., on an interior or exterior of the wall).

A particle comprising a gel component and a walled component may comprise multiple layers. In some cases, the gel is a first layer and the walled component is or approximates a second layer of the particle. In other cases, the walled component is a first layer and the gel is or approximates a second layer of the particle. The first layer may be partially or completely encompassed or surrounded by the second layer, or vice versa. In an example, the first layer may be a center (e.g., core) of the particle comprising the gel and the second layer may be a walled component coating on the first layer. In this instance, the second and first layers may be substantially concentric. In some cases, the walled component may encapsulate at least about 5%, 10%, 15%, 20%, 25%, 30%, 35%, 40%, 45%, 50%, 55%, 60%, 65%, 70%, 75%, 80%, 85%, 90%, 95%, 97%, 98%, or 99% of the gel. In some such cases, a walled component may comprise one or more components that partially or completely surround the inner gel, such as an aqueous solution comprising one or more analytes and/or reagents. In another example, the first layer may comprise the walled component and be a center (e.g., core) of the particle and the second layer comprising the gel may be a coating on the first layer (e.g., walled component). The gel may encapsulate at least about 5%, 10%, 15%, 20%, 25%, 30%, 35%, 40%, 45%, 50%, 55%, 60%, 65%, 70%, 75%, 80%, 85%, 90%, 95%, 97%, 98%, or 99% of the walled component.

A particle may comprise one or more cores (e.g., one or more approximately central locations about which one or more layers of gels and/or walled components may be disposed). A particle core may comprise a semi-fluidic (e.g., mixture of solid and liquid, or semi-solid) or fluidic material. For example, the particle may comprise a first layer comprising a walled component having a wall that encapsulates a solid, liquid, or semi-fluid core, and the gel may be disposed partially or completely on or around the walled component. Alternatively, the particle may comprise a first layer comprising a gel (e.g., a solid, semi-solid, fluidic, or semi-fluidic gel) at its core and the walled component may partially or completely encapsulate the gel. In such cases, the walled component may be described as comprising a bead (e.g., as described herein).

A gel or a wall of a walled component may be formed by polymerization of polymeric precursors within a droplet, as described elsewhere herein. For example, the gel may be formed by providing polymeric precursors (e.g., monomers) within a droplet and subjecting the droplet to a stimulus (e.g., ultraviolet light) to induce polymerization or crosslinking. A wall of a walled component may be similarly formed. In some cases, a wall of a walled component may be formed by providing polymeric precursors within a partition (e.g., a droplet or well) including the gel comprising a first biological material (e.g., an analyte or a reagent) and subjecting the partition to a stimulus to induce polymerization. The wall may form around or adjacent to the gel, as described herein. In some cases, the wall may partially or completely encapsulate one or more fluid or semi-fluid materials (e.g., an aqueous solution) that partially or completely surround the gel. In some cases, the fluid or semi-fluid contents of a walled component may at least partially permeate into the gel layer. The walled component may comprise a second biological material (e.g., an analyte or reagent) disposed on or embedded within a wall or contained within the walled component. In other cases, the gel comprising a first biological material (e.g., an analyte or a reagent) may be formed by polymerizing polymeric precursors within a droplet including the walled component such that the gel forms around or adjacent to the walled component. The walled component may comprise a second biological material (e.g., an analyte or reagent) disposed on or embedded within a wall or contained within the walled component. In some cases, the gel may be formed by providing polymeric precursors within a partition (e.g., a droplet or well) including the walled component comprising a first biological material (e.g., an analyte or a reagent) and subjecting the partition to a stimulus to induce polymerization. The gel may form around or adjacent to the walled component, as described herein. The gel may comprise a second biological material (e.g., an analyte or reagent). In other cases, the walled component comprising a first biological material (e.g., an analyte or a reagent) may be formed by polymerizing polymeric precursors within a droplet including the gel such that the walled component forms around or adjacent to the gel. The gel may comprise a second biological material (e.g., an analyte or reagent).

Particles including one or more gel components and one or more walled components may be formed according to the droplet generation methods described elsewhere herein. During formation of a gel or walled component, a biological material may undergo a chemical or physical change. For example, a biological material may undergo crosslinking with a polymerizable material used to form a gel or walled component. Accordingly, a gel or walled component may include a derivative of a biological material.

The particle may include at least 1, 2, 3, 4, 5, 6, 7, 8, 9, 10, 15, 20, 25, 30, 35, 40, 45, 50, 55 60, 65, 70, 75, 80, 85, 90, 95, 100, or more gels. One or more gels included in a multi-gel particle may comprise the same material (e.g., be formed of the same polymeric precursors). Gels comprising the same material may be separately formed and/or separately disposed within a particle. For example, a particle may include two gels (e.g., gel components) comprising the same material (e.g., a first layer including an analyte and a second layer formed of the same material and including one or more reagents). Alternatively, a particle may include two or more gels comprising different materials. In some cases, a particle may include alternating gel layers comprising two or more materials. For example, a particle may include a first layer comprised of a first material, a second layer comprised of a second material that is different from the first material, a third layer comprised of a third material that is the same as the first material, a fourth layer comprised of a fourth material that is the same as the second material, etc. Alternatively, each gel of a multi-gel particle may comprise a different material. Such gels may be situated in separate or discrete layers. The polymeric precursors used to form each gel may be as described elsewhere herein. As described herein, gels formed of different materials or comprised of the same materials but having different thicknesses and/or contents associated therewith may have different properties, including different susceptibilities to various stimuli. For example, a first gel formed of a first material may degrade or dissolve at a faster rate than a second gel formed of a second material that is different than the first material when the gels are exposed to the same stimulus. The disposition of the gels within the particle may also affect their response to stimuli. For example, a first gel disposed around a second gel may be more susceptible to a stimulus than the second gel due at least in part to the enhanced availability of the first gel to the stimulus. Upon dissolution or degradation of the first gel disposed around it, the second gel may become more susceptible to that stimulus or another stimulus.

Similarly, the particle may include at least 1, 2, 3, 4, 5, 6, 7, 8, 9, 10, 15, 20, 25, 30, 35, 40, 45, 50, 55 60, 65, 70, 75, 80, 85, 90, 95, 100, or more walled components. One or more walled components included in a particle may comprise walls formed of the same material (e.g., be formed of the same polymeric precursors). Alternatively, one or more walled components included in a particle may comprise walls formed of different materials (e.g., different polymeric precursors). As described herein, walls formed of different materials or comprised of the same materials but having different thicknesses and/or contents associated therewith may have different properties, including different susceptibilities to various stimuli. For example, a first wall of a first walled component formed of a first material may degrade or dissolve at a faster rate than a second wall of a second walled component formed of a second material that is different than the first material when the walled component are exposed to the same stimulus. The disposition of walls within the particle may also affect their response to stimuli. For example, a first wall disposed about a second wall of a walled component may be more susceptible to a stimulus than the second wall due at least in part to the enhanced availability of the first wall to the stimulus. Upon dissolution or degradation of the first wall disposed around it, the second wall may become more susceptible to that stimulus or another stimulus.

In an example, a particle may include two walled components comprising walls formed of the same material (e.g., a first walled component comprising an analyte and a second walled component having a wall formed of the same material and comprising one or more reagents). Alternatively, a particle may include two or more walled components comprising walls formed of different materials. One or more walled components included in a particle may comprise interior components (e.g., fluidic or semi-fluidic contents) formed of the same material and/or comprising the same analytes and/or reagents. Walled components comprising the same or different materials may be separately formed and/or separately disposed within a particle. In some cases, a particle may include alternating walled components comprising different materials. For example, a particle may include a first walled component comprised of a first material or having a first material encapsulated therein, a second walled component comprised of a second material that is different from the first material or having a second material encapsulated therein that is different from the first material, a third walled component comprised of a third material that may be the same as the first material or having a third material encapsulated therein, etc. In some cases, each walled component of a particle may comprise a wall formed of the same material but have one or more different contents. Alternatively, each walled component of a particle may comprise a wall formed of a different material and/or have different contents. Such walled components may be situated in separate or discrete layers. In some cases, walled components may be concentrically disposed within a particle. One or more walled components may encapsulate a gel layer. Alternatively or in addition, one or more walled components may comprise one or more gel layers disposed thereon. One or more gel layers may partially or completely separate one or more walled components. For example, one or more walled components may be disposed partially or completely within, or partially or completely around, one or more gels. The polymeric precursors used to form each walled component may be as described elsewhere herein.

In an example, a particle may comprise a walled component disposed at its core, which walled component is partially or completely encapsulated by a gel layer. In another example, a particle may comprise a walled component disposed at its core, which walled component is partially or completely encapsulated by two or more gel layers. In another example, a particle may comprise a walled component disposed at its core, which walled component is partially or completely encapsulated by one or more gel layers, which one or more gel layers are partially or completely encapsulated by an additional walled component. In another example, a particle may comprise a walled component disposed at its core, which walled component is partially or completely encapsulated by one or more gel layers, which one or more gel layers are partially or completely encapsulated by an additional walled component, which additional walled component is partially or completely encapsulated by one or more additional gel layers.

In another example, a particle may comprise one or more gels disposed at its core, which one or more gels are partially or completely encapsulated by a walled component. In another example, a particle may comprise one or more gels disposed at its core, which one or more gels are partially or completely encapsulated by a walled component, which walled component is partially or completely encapsulated by one or more additional gels. In another example, a particle may comprise one or more gels disposed at its core, which one or more gels are partially or completely encapsulated by a walled component, which walled component is partially or completely encapsulated by one or more additional gels, which one or more additional gels are partially or completely encapsulated by an additional walled component.

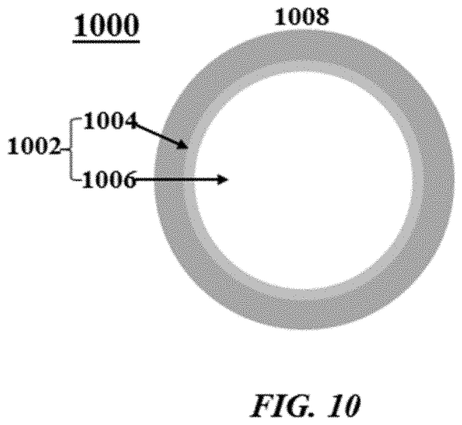

FIG. 10 shows an example of a particle 1000 including a gel component 1008 and a walled component 1002. The walled component 1002 comprises wall 1004 and interior region 1006. Wall 1004 may be formed from a polymerized material (e.g., as described herein) and may be porous or impermeable. For example, wall 1004 may be substantially impermeable to at least the contents of interior region 1006 such that the contents remain in interior region 1006. Interior region 1006 may comprise an aqueous solution comprising a biological material, such as an analyte or reagent. Alternatively or in addition, wall 1004 may comprise a biological material entrained therein or coupled to a surface thereof. Gel component 1008 may at least partially encompass walled component 1002. For example, gel component 1008 and walled component 1002 may be substantially concentric. Gel component 1008 may comprise a biological material, such as an analyte or reagent. The biological material may or may not be in contact with walled component 1002. Gel component 1008 and walled component 1002 may be dissolvable or disruptable upon application of the same or different stimuli.

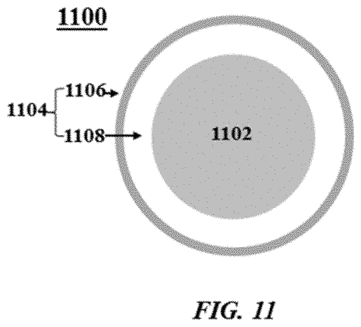

FIG. 11 shows an example of a particle 1100 including a gel component 1102 and a walled component 1104. Gel component 1102 may comprise a biological material, such as an analyte or reagent. The biological material may be distributed throughout the gel or coupled to a surface of gel component 1102 (e.g., as described herein). Gel component 1102 may be fluid, semi-fluid, semi-solid, or solid. In an example, gel component 1102 may be a gel bead. Walled component 1104 comprises wall 1106 and interior region 1108. Wall 1106 may be formed from a polymerized material (e.g., as described herein) and may be porous or impermeable. For example, wall 1106 may be substantially impermeable to at least the contents of interior region 1108 such that the contents remain in interior region 1108. Interior region 1108 may comprise an aqueous solution comprising a biological material, such as an analyte or reagent. Alternatively or in addition, wall 1106 may comprise a biological material entrained therein or coupled to a surface thereof. Walled component 1104 may at least partially encompass gel component 1102. For example, gel component 1102 and walled component 1104 may be substantially concentric. A biological material of gel component 1102 may or may not be in contact with walled component 1104. Gel component 1102 and walled component 1104 may be dissolvable or disruptable upon application of the same or different stimuli.

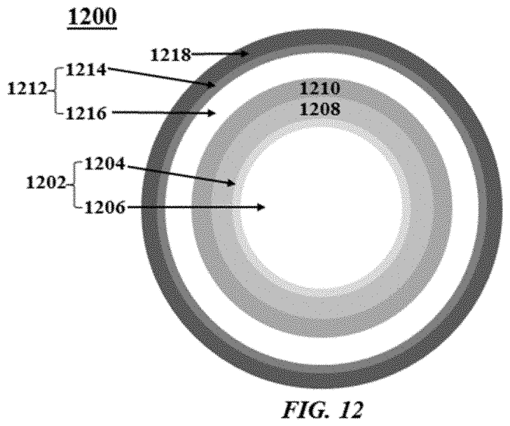

FIG. 12 shows an example of a particle 1200 including multiple gels and walled components. Walled component 1202 comprising wall 1204 and interior region 1206 is disposed at the core of particle 1200. Interior region 1206 may comprise a fluid such as an aqueous solution comprising a biological material, such as an analyte or reagent. Alternatively or in addition, wall 1204 may comprise a biological material entrained therein or coupled to a surface thereof. Walled component 1202 may be at least partially encompassed by gel components 1208 and 1210, which may each include a biological material (e.g., an analyte or reagent). Walled component 1212 comprising wall 1214 and interior region 1216 may at least partially encompass gel components 1208 and 1210 and walled component 1202. Interior region 1216 may comprise a fluid such as an aqueous solution comprising a biological material, such as an analyte or reagent. Alternatively or in addition, wall 1214 may comprise a biological material entrained therein or coupled to a surface thereof. Walled component 1212 may be at least partially encompassed by gel component 1218, which may comprise a biological material such as an analyte or reagent. The gel and walled components of particle 1200 may be substantially concentric. Biological materials of the gel and walled components of particle 1200 may be the same or different. For example, each gel component may comprise a reagent and each walled component may comprise an analyte such as a cell. The reagents of the gel components may be the same or different one another. The analytes of the walled components may derive from the same or different samples.

For a particle including multiple discrete layers of gels and/or walled components, each layer may have the same or a different thickness. For example, a particle may include a first inner layer (e.g., a core), a second layer surrounding the first layer, and a third layer surrounding the second layer. The second and third layers may have the same or different thickness. For a walled component, the thickness of a wall or combination of walls may be the same or different as the thickness of a wall or combination of walls for another component, and/or the thickness of the layer defined by the wall(s) may be the same or different as the thickness of the layer defined by the wall(s) of another walled component. Similarly, each gel (e.g., gel component) or walled component of the particle may have the same or different surface area. For particles including multiple discrete layers, the surface area of an inner layer (e.g., the core of the particle) may be smaller than the surface area of an outer layer (e.g., a first layer coating the core of the particle). Each gel or walled component of the particle may also have the same or different volumes. For example, the gel may have a larger volume (e.g., 1%, 5%, 10%, 15%, 20%, 30%, 50%, 75%, 100%, or a greater amount larger) than the walled component. Alternatively, the walled component may have a larger volume (e.g., 1%, 5%, 10%, 15%, 20%, 30%, 50%, 75%, 100%, or a greater amount larger) than the gel. Differences in thickness, surface area, and volume of different gel and walled components of a particle may result from, for example, different amounts or types of polymeric precursors used to form each gel and walled component, characteristics of each gel and capsule (e.g., water content, density, or tightness of packing), or contents of each gel and walled component (e.g., size and/or concentration of an analyte or reagent included therein and/or, for walled components, other components such as fluids included therein).

One or more gel or walled components of a particle may be substantially porous or substantially non-porous. A gel may be substantially solid, semi-solid, semi-fluidic, or fluidic. One or more components of a walled component may be substantially solid, semi-solid, semi-fluidic, or fluidic. For example, a wall of a walled component may be substantially solid or semi-solid and the interior contents of a walled component may be fluidic or semi-fluidic. In some cases, a walled component may encapsulate a fluidic or semi-fluidic material as well as a solid or semi-solid material, such as a gel (e.g., a gel bead). One or more gels or walled components of a particle may be rigid, flexible, and/or compressible. For example, a gel of a particle may be substantially flexible. In another example, a wall of a walled component may be substantially rigid. Alternatively, a wall of a walled component may be substantially flexible. Interior contents of a walled component may be at least somewhat compressible.

Properties of a gel or walled component or a combination of gels and/or walled component in a particle may be tailored based on a desired property or feature of a particle. For example, in cases in which (i) a gel substantially encompasses a walled component that comprises a biological material, (ii) a walled component substantially encompasses a gel and comprises a biological material, (iii) a gel substantially comprises a biological material and substantially encompasses a walled component, or (iv) a walled component substantially encompasses a gel that comprises a biological particle, properties of the gel and/or walled component may be tailored to regulate a rate at which the biological material becomes accessible. For example, for scenario (i), a thickness and chemical makeup of the gel may be selected such that a rate of dissolution of the gel impacts a time period within which disruption or dissolution of the wall of the walled component is initiated. For example, for scenario (iv), a thickness and chemical makeup of a wall of the walled component may be selected such that a rate of dissolution of the wall impacts a time period within which disruption or dissolution of the gel is initiated.

A particle comprising multiple gel and/or walled components may have any useful shape and size. For example, a particle may be spherical or substantially spherical, e.g., in the instance of a particle having multiple concentric or approximately concentric layers. Alternatively, a particle shape may be ovular, oblong, circular (e.g., disc-like), cylindrical, or amorphous. In one example, a particle may have a dumbbell shape. Such a particle may comprise two different entities defining each arm of the dumbbell. For example, such a particle may have a first gel component comprising a first cell and a second gel component comprising a second cell, where the first and second gel components meet or overlap between the two cells. Alternatively, such a particle may have a gel component comprising a cell and a walled component disposed approximately adjacent to the gel component. The walled component may encapsulate another cell and/or one or more analytes or reagents.

A particle may have a dimension (e.g., a diameter) that is at least about 1 nanometer (nm), 5 nm, 10 nm, 20 nm, 30 nm, 40 nm, 50 nm, 60 nm, 70 nm, 80 nm, 90 nm, 100 nm, 200 nm, 300 nm, 400 nm, 500 nm, 600 nm, 700 nm, 800 nm, 900 nm, 1 micrometer (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. Alternatively, a particle may have a dimension (e.g., a diameter) that is less than about 100 nm, 500 nm, lμm, 5 μm, 10 μm, 20 μm, 30 μm, 40 μm, 50 μm, 60 μm, 70 μm, 80 μm, 90 μm, 100 μm, 250 μm, 500 μm, or 1 mm, or less. In the case of a particle having a dumbbell shape, the diameter of the first arm of the dumbbell (e.g., first gel component comprising a cell) may be the same or different from the diameter of the second arm of the dumbbell (e.g., walled component or second gel component comprising an additional cell). For example, the diameter of the first arm of the dumbbell may be smaller than the diameter of the second arm of the dumbbell.

A particle comprising multiple gel and/or walled components may be used to analyze an analyte (e.g., an analyte of interest) using any useful reagent or combination of reagents, including but not limited to those described elsewhere herein. A particle comprising at least one gel component and at least one walled component may include a biological particle (e.g., a cell or cell bead) or a component thereof, such as a nucleic acid. For example, the particle may comprise a cell or cell bead encapsulated within a walled component. The analyte may also be, for example, a peptide, protein, lipid, transcription factor, receptor, antibody, metabolite, or nucleic acid molecule. Reagents for analyzing an analyte of interest (e.g., disposed anywhere within the particle) may be selected from the non-limiting group consisting of enzymes, fluorophores, oligonucleotides, primers, barcodes, nucleic acid barcode molecules (e.g., nucleic acid barcode molecules comprising one or more barcode sequences), buffers, deoxynucleotide triphosphates, detergents, reducing agents, chelating agents, oxidizing agents, nanoparticles, and antibodies. In some cases, one or more reagents are selected from the group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

An analyte may be disposed in any useful location within a particle. An analyte may be disposed within a gel component or a walled component. In some cases, a particle may not include an analyte. For example, the analyte may be contained within a centrally located gel (e.g., a first gel) surrounded by an outer layer (e.g., a walled component and/or a second gel) containing one or more reagents. Alternatively, an analyte may be contained within a gel layer (e.g., a first gel) that overlays a gel (e.g., a second gel) or walled component containing one or more reagents. In another example, an analyte may be contained within a centrally located walled component surrounded by an outer layer (e.g., a gel and/or additional walled component) containing one or more reagents. In a further example, an analyte may be contained within a walled component that overlays a gel containing one or more reagents.

Particles may also include more than one analyte for analyzing and processing. For example, a given gel or walled component may include multiple analytes. Alternatively, the gel may comprise a first analyte and the walled component may include a second analyte. Alternatively, a first gel may comprise a first analyte and a second gel may comprise a second analyte, and/or a first walled component may comprise a first analyte and a second walled component may comprise a second analyte. A second analyte may be the same or different from a first analyte. For example, a particle may include two or more analytes selected from the non-limiting group consisting of biological particles (e.g., cells or cell beads), nucleic acids, proteins, lipids, transcription factors, receptors, antibodies, metabolites, and peptides. In some cases, a particle may include multiple cells or cell beads (e.g., a dumbbell particle). Multiple cells or cell beads may be of the same or different type and/or may derive from the same or different subjects. In some cases, a particle may include multiple nucleic acids, which nucleic acids may derive from the same or different cells. For example, multiple nucleic acids from the same cell may be disposed within the same gel or walled component. In another example, multiple first nucleic acids from the same first cell may be disposed within the same first gel or walled component and multiple second nucleic acids from the same second cell may be disposed within the same second gel or walled component that is different than the first gel or walled component, where the first cell and second cell are different cells. The first and second cells may derive from the same or different samples (e.g., from the same or different subject, such as from the same or different patient).

Similarly, one or more reagents may be disposed in any useful location within a particle. For example, a gel may include an analyte and a first reagent, and a walled component may at least partially encompass the gel and include a second reagent. The first and second reagents may be the same or different from one another. In one example, the gel comprises an analyte that is a cell and a first reagent capable of lysing the cell to release a component of the cell, while the walled component comprises a second reagent useful for analyzing or processing the component of the cell. In another example, a walled component may include an analyte and a first reagent, and a gel may at least partially encompass the walled component and include a second reagent. The first and second reagents may be the same or different from one another. For example, the walled component comprises an analyte that is a cell and a first reagent capable of lysing the cell to release a component of the cell, while the gel comprises a second reagent useful for analyzing or processing the component of the cell. In some cases of the preceding examples, the first reagent and the cell may be separately disposed or fixated within the gel or walled component such that lysing of the cell does not occur. For example, the first reagent may be coupled to or embedded within a wall of the walled component while the cell may be disposed within the walled component. Application of an appropriate stimulus to disrupt or dissolve the gel or wall of the walled component (e.g., as described herein) may permit the first reagent to come into contact with the cell to release the component of the cell. The cellular component may then be available to a second reagent (e.g., of a separate gel or walled component) for analyzing or processing. In some cases, a separate gel or walled component comprising the second reagent must be disrupted or dissolved (e.g., by application of an appropriate stimulus) to make the second reagent available to the cellular component.

One or more gels or walled components (e.g., walls of walled components) of a particle may be at least partially disrupted or dissolved by application of a stimulus. For example, a first gel may be disruptable or dissolvable by application of a stimulus and a second gel may not be disruptable or dissolvable by application of a stimulus, or vice versa. In another example, a gel may be disruptable or dissolvable by application of a stimulus and a walled component (e.g., wall of a walled component) may not be disruptable or dissolvable by application of a stimulus, or vice versa. Application of a stimulus may disrupt or dissolve one or more gels and/or walled components (e.g., walls of walled components) of a particle. In some cases, multiple gels and/or walls of a particle may be disrupted or dissolved by the same stimulus simultaneously or sequentially (e.g., one after another). For example, a stimulus may disrupt or dissolve a gel that substantially encompasses a walled component and subsequently disrupt or dissolve the walled component, or a stimulus may disrupt or dissolve a walled component that substantially encompasses a gel (e.g., a gel bead) and subsequently disrupt or dissolve the gel. Such a stimulus may be, for example, a chemical agent requiring a single application (e.g., introduction). The chemical agent may first interact with, and consequently disrupt or dissolve, a first component (e.g., a gel component or walled component), and then, subsequent to the disruption or dissolution of the first component, interact with a second component (e.g., a gel component or walled component). In another example, such a stimulus may be a photo-stimulus or thermal stimulus that requires multiple applications to disrupt or dissolve a gel and/or walled component. The first application of a photo-stimulus or thermal stimulus may disrupt or dissolve all or a portion of a component (e.g., a gel component or walled component), and a subsequent application of the photo-stimulus or thermal stimulus may disrupt or dissolve all or a portion of another component (e.g., a gel component or walled component). Alternatively, a separate stimulus may be necessary for the disruption or dissolution of each component of a particle. A stimulus may be selected from the non-limiting group consisting of chemical triggers, bulk changes, biological triggers, light triggers, thermal triggers, magnetic triggers, and any combination thereof. A stimulus may be a change in pH, a change in ion concentration, or a reducing agent. For example, a stimulus useful for disrupting or dissolving a gel component of a multi-component particle may be dithiothreitol. In one example, a first component (e.g., gel component or walled component) comprises an analyte and is surrounded by the second component (e.g., gel component or walled component) comprising a reagent and the first component is capable of disruption or dissolution by changing pH or an ion concentration. In another example, the first component (e.g., gel component or walled component) comprises an analyte and is surrounded by the second component (e.g., gel component or walled component) comprising a reagent and the second component (e.g., gel component or walled component) is capable of disruption or dissolution by changing pH or an ion concentration. In yet another example, the first component (e.g., gel component or walled component) comprises an analyte and is surrounded by the second component (e.g., gel component or walled component) comprising a reagent and the second component (e.g., gel component or walled component) is capable of disruption or dissolution by exposure to dithiothreitol.

In some examples, a stimulus is a chemical or biological stimulus included in the particle (e.g., a reducing agent in an outer layer of the particle). In such a case, the outer layer may be disrupted upon application of another stimulus (e.g., light) to release the chemical or biological stimulus, for example.

In another aspect, the present disclosure provides a method of forming a particle for use in processing or analyzing an analyte from a sample, comprising providing a first component (e.g., gel component or walled component) comprising a first biological material; generating a partition (e.g., a droplet or well) comprising the first component, a polymerizable material, and a second biological material; and subjecting the polymerizable material to conditions sufficient to form a second component (e.g., gel component or walled component). The second component may be separate from the first component. For example, the first component and the second component may be comprised of different materials, have been prepared according to different processes and/or at different times, and/or be physically separated from one another (e.g., with the first component encompassing the second component as a distinct layer or vice versa). For example the first component may be distinct from the second component. In some cases, the second component at least partially encompasses the first component. The first biological material may comprise the analyte or a reagent for processing or analyzing the analyte. Similarly, the second biological material may comprise the analyte or a reagent for processing or analyzing the analyte. At least one of the first component and the second component may comprise the analyte. Alternatively, neither the first component nor the second component may comprise the analyte. At least one of the first component and the second component may comprise a reagent for processing or analyzing the analyte. For example, the first component may comprise the analyte while the second component comprises one or more reagents for processing or analyzing the analyte. In another example, the first component comprises the analyte and at least one reagent while the second component comprises another reagent that is the same or different from a reagent in the first component. In a further example, the first component and the second component each comprise a reagent for processing or analyzing an analyte. At least one of the first component and the second component may be a gel, and at least one of the first component and the second component may be a walled component.

The method of forming a particle comprising a gel component and a walled component may be used to form any multi-component particle described herein.

The gel and walled components of the particle may be formed as described herein (e.g., using microfluidics methods, air knife droplet generation, aerosol generation, or a membrane based encapsulation system). Generating the partition (e.g., droplet) comprising the gel, a polymerizable material, and a biological material (e.g., an analyte and/or one or more reagents for processing or analyzing an analyte) may comprise flowing (i) a first phase comprising an aqueous fluid, the polymerizable material, and the biological material and (ii) a second phase comprising a fluid that is immiscible with the aqueous fluid toward a junction. Upon interaction of the first and second phases, a discrete droplet of the first phase may be formed. The polymerizable material may then be subjected to a stimulus capable of polymerizing it into a gel or polymer that may be a wall of a walled component. Alternatively, a partition comprising a walled component, polymerizable material, and a biological material may be generated by flowing the first phase and the second phase toward a junction to interact the first and second phases and form a discrete droplet of the first phase. The polymerizable material may then be subjected to a stimulus capable of polymerizing it into a gel that may at least partially encompass the walled component. The stimulus may be selected from, for example, thermal stimuli (e.g., heating or cooling), photo-stimuli (e.g., through photo-curing), chemical stimuli (e.g., through crosslinking or added initiators), and any combination thereof. The stimulus capable of polymerizing the polymerizable material into a gel may be a material included in the first phase. Such a stimulus may be capable of polymerizing the polymerizable material into a gel or polymeric wall in situ. The polymerizing may comprise subunit addition and/or cross-linking. As described elsewhere herein, generation of a walled component may comprise generating a first droplet comprising a first phase and generating a second droplet comprising a second phase around the first droplet, where the second droplet comprises a polymerizable material. The polymerizable material in the second droplet may then be polymerized (e.g., by application of a stimulus) to generate a wall of a walled component. The interior contents of the walled component may comprise the first droplet.

The particle generation method described herein may comprise generating an additional droplet comprising a gel and walled component, two walled components, or two separate gels. The additional droplet may comprise an additional polymerizable material that may be subjected to conditions sufficient to form an additional gel or walled component separate from the other components of the particle. The additional gel or walled component may at least partially encompass the other components of the particle (e.g., the gel(s) and/or walled component(s)). These steps may be repeated one or more times to form a particle including two or more (e.g., three, four, five, six, seven, eight, nine, ten, or more) gel components and at least one walled component, or two or more (e.g., three, four, five, six, seven, eight, nine, ten, or more) walled components and at least one gel component. Any gel or walled component may include a biological material (e.g., an analyte or a reagent for processing or analyzing an analyte). One or more gel or walled components may be formed of the same polymerizable material, as described herein. Alternatively or in addition, one or more gel or walled components may be formed of different polymerizable materials. In one example, a particle may include a first gel and a third gel formed of the same polymerizable material and a walled component comprising a wall formed of a different polymerizable material, e.g., in an alternating pattern.

A particle comprising a gel component and a walled component may comprise multiple layers (e.g., as described herein). In some cases, the gel is a first layer and the walled component is or approximates a second layer of the particle. In other cases, the walled component is a first layer and the gel is or approximates a second layer of the particle. The first layer may be partially or completely encompassed or surrounded by the second layer, or vice versa. For example, the first layer may be a center (e.g., core) of the particle comprising the gel and the second layer may be a walled component coating on the first layer. Such layers may be substantially concentric. In some cases, the walled component may encapsulate at least about 5%, 10%, 15%, 20%, 25%, 30%, 35%, 40%, 45%, 50%, 55%, 60%, 65%, 70%, 75%, 80%, 85%, 90%, 95%, 97%, 98%, or 99% of the gel. In some such cases, a walled component may comprise one or more components that partially or completely surround the inner gel, such as an aqueous solution comprising one or more analytes and/or reagents. In another example, the first layer may comprise the walled component and be a center (e.g., core) of the particle and the second layer comprising the gel may be a coating on the first layer (e.g., walled component). The gel may encapsulate at least about 5%, 10%, 15%, 20%, 25%, 30%, 35%, 40%, 45%, 50%, 55%, 60%, 65%, 70%, 75%, 80%, 85%, 90%, 95%, 97%, 98%, or 99% of the walled component.

An analyte may be included in either the gel or the walled component, as described herein. In some cases, multiple components of a particle may include an analyte. For example, both the gel and the walled component may include an analyte. These analytes may be the same or different. Analytes may be, for example, biological particles (e.g., cells) or components thereof, nucleic acids, peptides, proteins, lipids, transcription factors, receptors, antibodies, metabolites, or any other analyte of interest. In certain cases, one or more components of a particle may include a cell. In an example, a gel may comprise a first cell and a walled component may comprise a second cell, which first and second cells derive from the same sample (e.g., the same subject). In another example, a gel may comprise a first cell and a walled component may comprise a second cell, which first and second cells derive from different samples (e.g., different samples taken from the same subject at different times, different samples taken from the same subject by different mechanisms (e.g., blood sample vs. other fluid sample), or different samples taken from different subjects).

At least one of the gel and the walled component may include one or more reagents, as described herein. The gel may comprise an analyte and one or more reagents may be included in the walled component. Alternatively, the gel may comprise one or more reagents and the walled component may comprise an analyte. In other cases, both the gel and the walled component may include analytes, or the gel and the walled component may both include reagents. Particles may include one or more reagents in multiple gel and/or walled components. For example, a particle may have two gel components that each include at least one reagent, two walled components that each include at least one reagent, and/or a gel and a walled component that both include at least one reagent. Reagents included in different components of a particle may be the same or different. A reagent included in a particle made by a method of the present disclosure may be any useful reagent to achieve any useful purpose toward analyzing and processing an analyte. One or more reagents may be selected from the non-limiting group consisting of enzymes, fluorophores, oligonucleotides, primers, barcodes, buffers, deoxynucleotide triphosphates, detergents, reducing agents, chelating agents, oxidizing agents, nanoparticles, and antibodies. One or more reagents may also be selected from the non-limiting group consisting of temperature-sensitive enzymes, pH-sensitive enzymes, light-sensitive enzymes, reverse transcriptase, proteases, ligase, polymerases, restriction enzymes, transposase, nucleases, protease inhibitors, and nuclease inhibitors.

At least one gel or walled component of a particle formed by the methods disclosed herein may be disruptable or dissolvable upon application of a stimulus, as described herein. In some cases, the gel may be disruptable or dissolvable upon application of a stimulus. In other cases, the walled component (e.g., wall of the walled component) may be disruptable or dissolvable upon application of a stimulus. A stimulus capable of disrupting or dissolving one or more gel and/or walled components of a particle may be selected from the non-limiting group consisting of chemical triggers, bulk changes, biological triggers, light triggers, thermal triggers, magnetic triggers, and any combination thereof. In some instances, a stimulus may be selected from the non-limiting group consisting of a change in pH, a change in ion concentration, and a reducing agent such as dithiothreitol.

In another aspect, the present disclosure provides a kit including a plurality of particles each comprising (i) a gel comprising a first biological material (e.g., an analyte or a reagent), and (ii) a walled component comprising a second biological material (e.g., an analyte or a reagent). For example, a kit may include an array of particles (e.g., 2, 4, 6, 8, 10, 20, 40, 60, 80, 100, 200, 300, 400, 500, 1000, or more particles). The gel and walled components may be separate from one another. For example, the gel and the walled component may be comprised of different materials, have been prepared according to different processes and/or at different times, and/or be physically separated from one another (e.g., with the gel encompassing the walled component as a distinct layer or vice versa). For example the gel may be distinct from the walled component. The particles included in a kit may be the same (e.g., comprising the same gels, analytes, reagents, and configuration) or different. The walled component of a given particle may at least partially encompass the gel. Alternatively, the gel of a given particle may at least partially encompass the walled component. The gel of each particle of the plurality of particles may be formed of the same polymerizable material. The walled component of each particle of the plurality of particles may also or alternatively be formed of the same polymerizable material. The polymerizable material of the gel may be the same or different from the polymerizable material of the walled component. The first biological material and/or the second biological material may comprise an analyte, such as a cell or nucleic acid. The particles may each include a cell. The cells may derive from the same organism or a component thereof (e.g., a tissue) or the same cell line or may derive from different sources. Alternatively, the particles may each include a nucleic acid that is the same or different in each particle.

The first biological material and/or the second biological material may also or alternatively comprise a reagent for processing or analyzing an analyte. A particle may include one or more reagents disposed in the same or another component (e.g., gel or walled component). A reagent may comprise a nucleic acid barcode molecule that may include a barcode sequence. In one example, each particle of the plurality of particles may comprise a nucleic acid barcode molecule including a barcode sequence. Each particle of the plurality of particles may include a different barcode sequence. A particle of the plurality of particles may include a plurality of nucleic acid barcode molecules, where each of the plurality of nucleic acid barcode molecules includes a barcode sequence, where the barcode sequence is the same for each nucleic acid barcode molecules of the plurality of nucleic acid barcode molecules. Each nucleic acid barcode molecule of the plurality of nucleic acid barcode molecules may also include a second barcode sequence. This second barcode sequence may vary between the nucleic acid barcode molecules of the plurality of nucleic acid barcode molecules associated with a given particle. For example, all or a subset of the plurality of nucleic acid barcode molecules associated with a given particle may have a different second barcode sequence.

In another aspect, the present disclosure provides a kit including a plurality of particles each comprising a gel or walled component and a polymerizable material. Such a kit may include, for example, an array of particles (e.g., 2, 4, 6, 8, 10, 20, 40, 60, 80, 100, 200, 300, 400, 500, 1000, or more particles) each including a single gel or walled component. The single gel or walled component of each particle of the plurality of particles may be comprised of the same polymerizable material. The particles of the plurality of particles may include a biological material such as an analyte or a reagent for processing or analyzing an analyte. The analyte may be a cell, a nucleic acid, protein, lipid, transcription factor, receptor, antibody, metabolite, or a peptide, as described herein. The reagent may comprise a nucleic acid barcode molecule that may include a barcode sequence. In one example, each particle of the plurality of particles may comprise a nucleic acid barcode molecule including a barcode sequence. Each particle of the plurality of particles may include a different barcode sequence. A particle of the plurality of particles may include a plurality of nucleic acid barcode molecules, where each of the plurality of nucleic acid barcode molecules includes a barcode sequence, where the barcode sequence is the same for each nucleic acid barcode molecules of the plurality of nucleic acid barcode molecules. Each nucleic acid barcode molecule of the plurality of nucleic acid barcode molecules may also include a second barcode sequence. This second barcode sequence may vary between the nucleic acid barcode molecules of the plurality of nucleic acid barcode molecules associated with a given particle. For example, all or a subset of the plurality of nucleic acid barcode molecules associated with a given particle may have a different second barcode sequence. The particles may also include one or more additional reagents.

The polymerizable material provided in the kit may be the same as or different from that used to form the first gel or walled component of the particles. The kit may be provided with one or more cells or nucleic acids, or with one or more reagents. Such a kit may be used according to the methods disclosed herein. For example, a particle of the kit comprising a single gel or walled component comprising a nucleic acid barcode molecule including a barcode sequence may be combined in a partition (e.g., a droplet) with a polymerizable material and an analyte of interest (e.g., a cell or nucleic acid that may or may not be included with the kit). The polymerizable material may then be subjected to conditions sufficient to form a second gel or walled component separate from the first gel or walled component that includes the analyte of interest. The particle created thereby may be used, e.g., as described elsewhere herein.

### Systems and Methods for Sample Compartmentalization

In an aspect, the systems and methods described herein provide for the compartmentalization, depositing, or partitioning of macromolecular constituent contents of individual biological particles into discrete compartments or partitions (referred to interchangeably herein as partitions), where each partition maintains separation of its own contents from the contents of other partitions. The partition can be a droplet in an emulsion. A partition may comprise one or more other partitions.

A partition of the present disclosure may comprise biological particles and/or macromolecular constituents thereof. A partition may comprise one or more gel beads. A partition may comprise one or more cell beads. A partition may include a single gel bead, a single cell bead, or both a single cell bead and single gel bead. A cell bead can be a biological particle and/or one or more of its macromolecular constituents encased inside of a gel or polymer matrix, such as via polymerization of a droplet containing the biological particle and precursors capable of being polymerized or gelled. Unique identifiers, such as barcodes, may be injected into the droplets previous to, subsequent to, or concurrently with droplet generation, such as via a microcapsule (e.g., bead), as described further below. Microfluidic channel networks (e.g., on a chip) can be utilized to generate partitions as described herein. Alternative mechanisms may also be employed in the partitioning of individual biological particles, including porous membranes through which aqueous mixtures of cells are extruded into non-aqueous fluids.

The partitions can be flowable within fluid streams. The partitions may comprise, for example, micro-vesicles that have an outer barrier surrounding an inner fluid center or core. In some cases, the partitions may comprise a porous matrix that is capable of entraining and/or retaining materials within its matrix. The partitions can comprise droplets of aqueous fluid within a non-aqueous continuous phase (e.g., oil phase). The partitions can comprise droplets of a first phase within a second phase, wherein the first and second phases are immiscible. A variety of different vessels are described in, for example, U.S. Patent Application Publication No. 2014/0155295, which is entirely incorporated herein by reference for all purposes. Emulsion systems for creating stable droplets in non-aqueous or oil continuous phases are described in, for example, U.S. Patent Application Publication No. 2010/0105112, which is entirely incorporated herein by reference for all purposes.

In the case of droplets in an emulsion, allocating individual biological particles to discrete partitions may in one non-limiting example be accomplished by introducing a flowing stream of biological particles in an aqueous fluid into a flowing stream of a non-aqueous fluid, such that droplets are generated at the junction of the two streams. By providing the aqueous stream at a certain concentration and/or flow rate of biological particles, the occupancy of the resulting partitions (e.g., number of biological particles per partition) can be controlled. Where single biological particle partitions are used, the relative flow rates of the immiscible fluids can be selected such that, on average, the partitions may contain less than one biological particle per partition in order to ensure that those partitions that are occupied are primarily singly occupied. In some cases, partitions among a plurality of partitions may contain at most one biological particle (e.g., bead, cell or cellular material). In some embodiments, the relative flow rates of the fluids can be selected such that a majority of partitions are occupied, for example, allowing for only a small percentage of unoccupied partitions. The flows and channel architectures can be controlled as to ensure a given number of singly occupied partitions, less than a certain level of unoccupied partitions and/or less than a certain level of multiply occupied partitions.

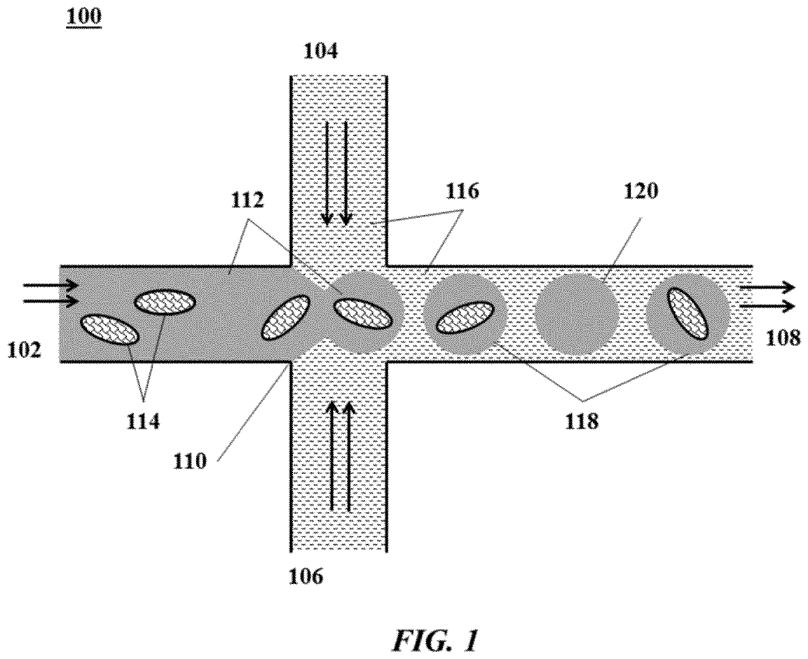

FIG. 1 shows an example of a microfluidic channel structure 100 for partitioning individual biological particles. The channel structure 100 can include channel segments 102, 104, 106 and 108 communicating at a channel junction 110. In operation, a first aqueous fluid 112 that includes suspended biological particles (or cells) 114 may be transported along channel segment 102 into junction 110, while a second fluid 116 that is immiscible with the aqueous fluid 112 is delivered to the junction 110 from each of channel segments 104 and 106 to create discrete droplets 118, 120 of the first aqueous fluid 112 flowing into channel segment 108, and flowing away from junction 110. The channel segment 108 may be fluidically coupled to an outlet reservoir where the discrete droplets can be stored and/or harvested. A discrete droplet generated may include an individual biological particle 114 (such as droplets 118). A discrete droplet generated may include more than one individual biological particle 114 (not shown in FIG. 1). A discrete droplet may contain no biological particle 114 (such as droplet 120). Each discrete partition may maintain separation of its own contents (e.g., individual biological particle 114) from the contents of other partitions.

The second fluid 116 can comprise an oil, such as a fluorinated oil, that includes a fluorosurfactant for stabilizing the resulting droplets, for example, inhibiting subsequent coalescence of the resulting droplets 118, 120. Examples of particularly useful partitioning fluids and fluorosurfactants are described, for example, in U.S. Patent Application Publication No. 2010/0105112, which is entirely incorporated herein by reference for all purposes.

As will be appreciated, the channel segments described herein may be coupled to any of a variety of different fluid sources or receiving components, including reservoirs, tubing, manifolds, or fluidic components of other systems. As will be appreciated, the microfluidic channel structure 100 may have other geometries. For example, a microfluidic channel structure can have more than one channel junction. For example, a microfluidic channel structure can have 2, 3, 4, or 5 channel segments each carrying biological particles, cell beads, and/or gel beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

The generated droplets may comprise two subsets of droplets: (1) occupied droplets 118, containing one or more biological particles 114, and (2) unoccupied droplets 120, not containing any biological particles 114. Occupied droplets 118 may comprise singly occupied droplets (having one biological particle) and multiply occupied droplets (having more than one biological particle). As described elsewhere herein, in some cases, the majority of occupied partitions can include no more than one biological particle per occupied partition and some of the generated partitions can be unoccupied (of any biological particle). In some cases, though, some of the occupied partitions may include more than one biological particle. In some cases, the partitioning process may be controlled such that fewer than about 25% of the occupied partitions contain more than one biological particle, and in many cases, fewer than about 20% of the occupied partitions have more than one biological particle, while in some cases, fewer than about 10% or even fewer than about 5% of the occupied partitions include more than one biological particle per partition.

In some cases, it may be desirable to minimize the creation of excessive numbers of empty partitions, such as to reduce costs and/or increase efficiency. While this minimization may be achieved by providing a sufficient number of biological particles (e.g., biological particles 114) at the partitioning junction 110, such as to ensure that at least one biological particle is encapsulated in a partition, the Poissonian distribution may expectedly increase the number of partitions that include multiple biological particles. As such, where singly occupied partitions are to be obtained, at most about 95%, 90%, 85%, 80%, 75%, 70%, 65%, 60%, 55%, 50%, 45%, 40%, 35%, 30%, 25%, 20%, 15%, 10%, 5% or less of the generated partitions can be unoccupied.

In some cases, the flow of one or more of the biological particles (e.g., in channel segment 102), or other fluids directed into the partitioning junction (e.g., in channel segments 104, 106) can be controlled such that, in many cases, no more than about 50% of the generated partitions, no more than about 25% of the generated partitions, or no more than about 10% of the generated partitions are unoccupied. These flows can be controlled so as to present a non-Poissonian distribution of single-occupied partitions while providing lower levels of unoccupied partitions. The above noted ranges of unoccupied partitions can be achieved while still providing any of the single occupancy rates described above. For example, in many cases, the use of the systems and methods described herein can create resulting partitions that have multiple occupancy rates of less than about 25%, less than about 20%, less than about 15%, less than about 10%, and in many cases, less than about 5%, while having unoccupied partitions of less than about 50%, less than about 40%, less than about 30%, less than about 20%, less than about 10%, less than about 5%, or less.

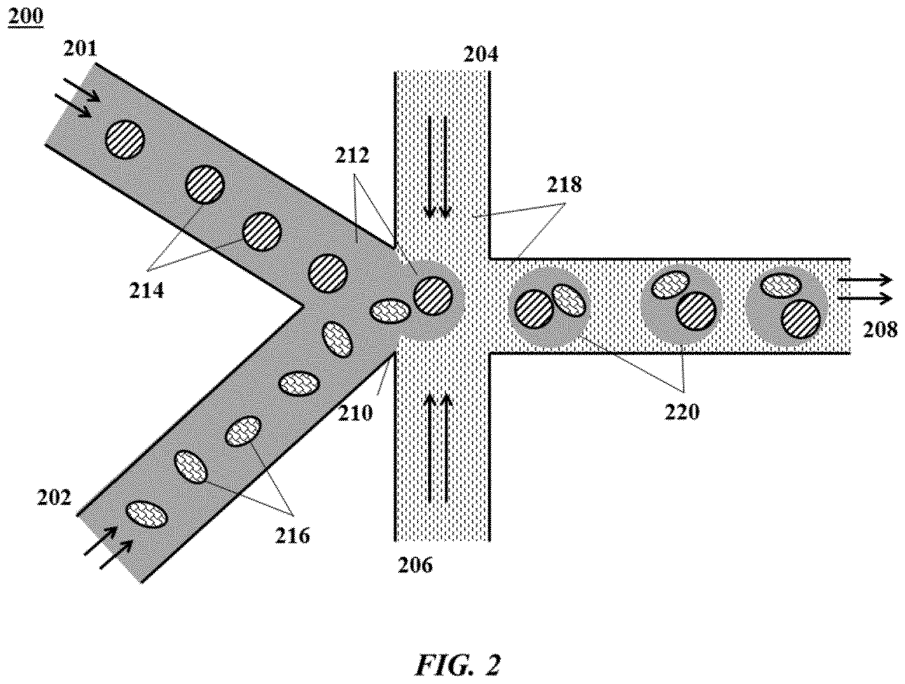

As will be appreciated, the above-described occupancy rates are also applicable to partitions that include both biological particles and additional reagents, including, but not limited to, microcapsules carrying barcoded nucleic acid molecules (e.g., oligonucleotides) (described in relation to FIG. 2). The occupied partitions (e.g., at least about 10%, 20%, 30%, 40%, 50%, 60%, 70%, 80%, 90%, 95%, or 99% of the occupied partitions) can include both a microcapsule (e.g., bead) comprising barcoded nucleic acid molecules and a biological particle.

In another aspect, in addition to or as an alternative to droplet based partitioning, biological particles may be encapsulated within a microcapsule that comprises an outer shell, layer or porous matrix in which is entrained one or more individual biological particles or small groups of biological particles. The microcapsule may include other reagents. Encapsulation of biological particles may be performed by a variety of processes. Such processes may combine an aqueous fluid containing the biological particles with a polymeric precursor material that may be capable of being formed into a gel or other solid or semi-solid matrix upon application of a particular stimulus to the polymer precursor. Such stimuli can include, for example, thermal stimuli (e.g., either heating or cooling), photo-stimuli (e.g., through photo-curing), chemical stimuli (e.g., through crosslinking, polymerization initiation of the precursor (e.g., through added initiators)), or a combination thereof. For example, an outer droplet may be formed around an inner droplet (e.g., according to the droplet generation methods provided herein) and the outer droplet may comprise a polymeric precursor material that may be capable of being formed into a gel or other solid or semi-solid matrix upon application of a particular stimulus to the polymeric precursor. Upon exposure to the stimulus, the polymeric precursor material may form a shell (e.g., wall) surrounding the inner droplet, which shell may be porous or impermeable, and which may be flexible or rigid. Such a method may be used to prepare a walled component (e.g., as described herein). In some cases, the inner droplet may include one or more analytes or reagents, such as one or more nucleic acids, cells, or reagents for processing the same. Similarly, the shell that forms around the inner droplet may comprise one or more analytes or reagents coupled thereto or embedded therein.

Preparation of microcapsules comprising biological particles may be performed by a variety of methods. For example, air knife droplet or aerosol generators may be used to dispense droplets of precursor fluids into gelling solutions in order to form microcapsules that include individual biological particles or small groups of biological particles. Likewise, membrane based encapsulation systems may be used to generate microcapsules comprising encapsulated biological particles as described herein. Microfluidic systems of the present disclosure, such as that shown in FIG. 1, may be readily used in encapsulating cells as described herein. In particular, and with reference to FIG. 1, the aqueous fluid 112 comprising (i) the biological particles 114 and (ii) the polymer precursor material (not shown) is flowed into channel junction 110, where it is partitioned into droplets 118, 120 through the flow of non-aqueous fluid 116. In the case of encapsulation methods, non-aqueous fluid 116 may also include an initiator (not shown) to cause polymerization and/or crosslinking of the polymer precursor to form the microcapsule that includes the entrained biological particles. Examples of polymer precursor/initiator pairs include those described in U.S. Patent Application Publication No. 2014/0378345, which is entirely incorporated herein by reference for all purposes.

For example, in the case where the polymer precursor material comprises a linear polymer material, such as a linear polyacrylamide, PEG, or other linear polymeric material, the activation agent may comprise a cross-linking agent, or a chemical that activates a cross-linking agent within the formed droplets. Likewise, for polymer precursors that comprise polymerizable monomers, the activation agent may comprise a polymerization initiator. For example, in certain cases, where the polymer precursor comprises a mixture of acrylamide monomer with a N,N′-bis-(acryloyl)cystamine (BAC) comonomer, an agent such as tetraethylmethylenediamine (TEMED) may be provided within the second fluid streams 116 in channel segments 104 and 106, which can initiate the copolymerization of the acrylamide and BAC into a cross-linked polymer network, or hydrogel.

Upon contact of the second fluid stream 116 with the first fluid stream 112 at junction 110, during formation of droplets, the TEMED may diffuse from the second fluid 116 into the aqueous fluid 112 comprising the linear polyacrylamide, which will activate the crosslinking of the polyacrylamide within the droplets 118, 120, resulting in the formation of gel (e.g., hydrogel) microcapsules, as solid or semi-solid beads or particles entraining the cells 114. Although described in terms of polyacrylamide encapsulation, other ‘activatable’ encapsulation compositions may also be employed in the context of the methods and compositions described herein. For example, formation of alginate droplets followed by exposure to divalent metal ions (e.g., Ca²⁺ ions), can be used as an encapsulation process using the described processes. Likewise, agarose droplets may also be transformed into capsules through temperature based gelling (e.g., upon cooling, etc.).

In some cases, encapsulated biological particles can be selectively releasable from the microcapsule, such as through passage of time or upon application of a particular stimulus, that degrades the microcapsule sufficiently to allow the biological particles (e.g., cell), or its other contents to be released from the microcapsule, such as into a partition (e.g., droplet). For example, in the case of the polyacrylamide polymer described above, degradation of the microcapsule may be accomplished through the introduction of an appropriate reducing agent, such as DTT or the like, to cleave disulfide bonds that cross-link the polymer matrix. See, for example, U.S. Patent Application Publication No. 2014/0378345, which is entirely incorporated herein by reference for all purposes.

The biological particle can be subjected to other conditions sufficient to polymerize or gel the precursors. The conditions sufficient to polymerize or gel the precursors may comprise exposure to heating, cooling, electromagnetic radiation, and/or light. The conditions sufficient to polymerize or gel the precursors may comprise any conditions sufficient to polymerize or gel the precursors. Following polymerization or gelling, a polymer or gel may be formed around the biological particle. The polymer or gel may be diffusively permeable to chemical or biochemical reagents. The polymer or gel may be diffusively impermeable to macromolecular constituents of the biological particle. In this manner, the polymer or gel may act to allow the biological particle to be subjected to chemical or biochemical operations while spatially confining the macromolecular constituents to a region of the droplet defined by the polymer or gel. The polymer or gel may include one or more of disulfide cross-linked polyacrylamide, agarose, alginate, polyvinyl alcohol, polyethylene glycol (PEG)-diacrylate, PEG-acrylate, PEG-thiol, PEG-azide, PEG-alkyne, other acrylates, chitosan, carboxymethylcellulose, hydroxypropyl methylcellulose, hyaluronic acid, collagen, fibrin, gelatin, or elastin. The polymer or gel may comprise any other polymer or gel.

The polymer or gel may be functionalized to bind to targeted analytes, such as nucleic acids, proteins, peptides, carbohydrates, lipids or other analytes. The polymer or gel may be polymerized or gelled via a passive mechanism. The polymer or gel may be stable in alkaline conditions or at elevated temperature. The polymer or gel may have mechanical properties similar to the mechanical properties of the bead. For instance, the polymer or gel may be of a similar size to the bead. The polymer or gel may have a mechanical strength (e.g. tensile strength) similar to that of the bead. The polymer or gel may be of a lower density than an oil. The polymer or gel may be of a density that is roughly similar to that of a buffer. The polymer or gel may have a tunable pore size. The pore size may be chosen to, for instance, retain denatured nucleic acids. The pore size may be chosen to maintain diffusive permeability to exogenous chemicals such as sodium hydroxide (NaOH) and/or endogenous chemicals such as inhibitors. The polymer or gel may be biocompatible. The polymer or gel may maintain or enhance cell viability. The polymer or gel may be biochemically compatible. The polymer or gel may be polymerized and/or depolymerized thermally, chemically, enzymatically, and/or optically.

The polymer may comprise poly(acrylamide-co-acrylic acid) crosslinked with disulfide linkages. The preparation of the polymer may comprise a two-step reaction. In the first activation step, poly(acrylamide-co-acrylic acid) may be exposed to an acylating agent to convert carboxylic acids to esters. For instance, the poly(acrylamide-co-acrylic acid) may be exposed to 4-(4,6-dimethoxy-1,3,5-triazin-2-yl)-4-methylmorpholinium chloride (DMTMM). The polyacrylamide-co-acrylic acid may be exposed to other salts of 4-(4,6-dimethoxy-1,3,5-triazin-2-yl)-4-methylmorpholinium. In the second cross-linking step, the ester formed in the first step may be exposed to a disulfide crosslinking agent. For instance, the ester may be exposed to cystamine (2,2′-dithiobis(ethylamine)). Following the two steps, the biological particle may be surrounded by polyacrylamide strands linked together by disulfide bridges. In this manner, the biological particle may be encased inside of or comprise a gel or matrix (e.g., polymer matrix) to form a “cell bead.” A cell bead can contain biological particles (e.g., a cell) or macromolecular constituents (e.g., RNA, DNA, proteins, etc.) of biological particles. A cell bead may include a single cell or multiple cells, or a derivative of the single cell or multiple cells. For example after lysing and washing the cells, inhibitory components from cell lysates can be washed away and the macromolecular constituents can be bound as cell beads. Systems and methods disclosed herein can be applicable to both cell beads (and/or droplets or other partitions) containing biological particles and cell beads (and/or droplets or other partitions) containing macromolecular constituents of biological particles.

Encapsulated biological particles can provide certain potential advantages of being more storable and more portable than droplet-based partitioned biological particles. Furthermore, in some cases, it may be desirable to allow biological particles to incubate for a select period of time before analysis, such as in order to characterize changes in such biological particles over time, either in the presence or absence of different stimuli. In such cases, encapsulation may allow for longer incubation than partitioning in emulsion droplets, although in some cases, droplet partitioned biological particles may also be incubated for different periods of time, e.g., at least 10 seconds, at least 30 seconds, at least 1 minute, at least 5 minutes, at least 10 minutes, at least 30 minutes, at least 1 hour, at least 2 hours, at least 5 hours, or at least 10 hours or more. The encapsulation of biological particles may constitute the partitioning of the biological particles into which other reagents are co-partitioned. Alternatively or in addition, encapsulated biological particles may be readily deposited into other partitions (e.g., droplets) as described above.

### Beads

A partition may comprise one or more unique identifiers, such as barcodes. Barcodes may be previously, subsequently or concurrently delivered to the partitions that hold the compartmentalized or partitioned biological particle. For example, barcodes may be injected into droplets previous to, subsequent to, or concurrently with droplet generation. The delivery of the barcodes to a particular partition allows for the later attribution of the characteristics of the individual biological particle to the particular partition. Barcodes may be delivered, for example on a nucleic acid molecule (e.g., an oligonucleotide), to a partition via any suitable mechanism. Barcoded nucleic acid molecules can be delivered to a partition via a microcapsule. A microcapsule, in some instances, can comprise a bead. Beads are described in further detail below.

In some cases, barcoded nucleic acid molecules can be initially associated with the microcapsule and then released from the microcapsule. Release of the barcoded nucleic acid molecules can be passive (e.g., by diffusion out of the microcapsule). In addition or alternatively, release from the microcapsule can be upon application of a stimulus which allows the barcoded nucleic acid nucleic acid molecules to dissociate or to be released from the microcapsule. Such stimulus may disrupt the microcapsule, an interaction that couples the barcoded nucleic acid molecules to or within the microcapsule, or both. Such stimulus can include, for example, a thermal stimulus, photo-stimulus, chemical stimulus (e.g., change in pH or use of a reducing agent(s)), a mechanical stimulus, a radiation stimulus; a biological stimulus (e.g., enzyme), or any combination thereof.

FIG. 2 shows an example of a microfluidic channel structure 200 for delivering barcode carrying beads to droplets. The channel structure 200 can include channel segments 201, 202, 204, 206 and 208 communicating at a channel junction 210. In operation, the channel segment 201 may transport an aqueous fluid 212 that includes a plurality of beads 214 (e.g., with nucleic acid molecules, oligonucleotides, molecular tags) along the channel segment 201 into junction 210. The plurality of beads 214 may be sourced from a suspension of beads. For example, the channel segment 201 may be connected to a reservoir comprising an aqueous suspension of beads 214. The channel segment 202 may transport the aqueous fluid 212 that includes a plurality of biological particles 216 along the channel segment 202 into junction 210. The plurality of biological particles 216 may be sourced from a suspension of biological particles. For example, the channel segment 202 may be connected to a reservoir comprising an aqueous suspension of biological particles 216. In some instances, the aqueous fluid 212 in either the first channel segment 201 or the second channel segment 202, or in both segments, can include one or more reagents, as further described below. A second fluid 218 that is immiscible with the aqueous fluid 212 (e.g., oil) can be delivered to the junction 210 from each of channel segments 204 and 206. Upon meeting of the aqueous fluid 212 from each of channel segments 201 and 202 and the second fluid 218 from each of channel segments 204 and 206 at the channel junction 210, the aqueous fluid 212 can be partitioned as discrete droplets 220 in the second fluid 218 and flow away from the junction 210 along channel segment 208. The channel segment 208 may deliver the discrete droplets to an outlet reservoir fluidly coupled to the channel segment 208, where they may be harvested.

As an alternative, the channel segments 201 and 202 may meet at another junction upstream of the junction 210. At such junction, beads and biological particles may form a mixture that is directed along another channel to the junction 210 to yield droplets 220. The mixture may provide the beads and biological particles in an alternating fashion, such that, for example, a droplet comprises a single bead and a single biological particle.

Beads, biological particles and droplets may flow along channels at substantially regular flow profiles (e.g., at regular flow rates). Such regular flow profiles may permit a droplet to include a single bead and a single biological particle. Such regular flow profiles may permit the droplets to have an occupancy (e.g., droplets having beads and biological particles) greater than 5%, 10%, 20%, 30%, 40%, 50%, 60%, 70%, 80%, 90%, or 95%. Such regular flow profiles and devices that may be used to provide such regular flow profiles are provided in, for example, U.S. Patent Publication No. 2015/0292988, which is entirely incorporated herein by reference.

The second fluid 218 can comprise an oil, such as a fluorinated oil, that includes a fluorosurfactant for stabilizing the resulting droplets, for example, inhibiting subsequent coalescence of the resulting droplets 220.

A discrete droplet that is generated may include an individual biological particle 216. A discrete droplet that is generated may include a barcode or other reagent carrying bead 214. A discrete droplet generated may include both an individual biological particle and a barcode carrying bead, such as droplets 220. In some instances, a discrete droplet may include more than one individual biological particle or no biological particle. In some instances, a discrete droplet may include more than one bead or no bead. A discrete droplet may be unoccupied (e.g., no beads, no biological particles).

Beneficially, a discrete droplet partitioning a biological particle and a barcode carrying bead may effectively allow the attribution of the barcode to macromolecular constituents of the biological particle within the partition. The contents of a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 200 may have other geometries. For example, a microfluidic channel structure can have more than one channel junctions. For example, a microfluidic channel structure can have 2, 3, 4, or 5 channel segments each carrying beads that meet at a channel junction. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

A bead may be porous, non-porous, solid, semi-solid, semi-fluidic, fluidic, and/or a combination thereof. In some instances, a bead may be dissolvable, disruptable, and/or degradable. In some cases, a bead may not be degradable. In some cases, the bead may be a gel bead. A gel bead may be a hydrogel bead. A gel bead may be formed from molecular precursors, such as a polymeric or monomeric species. A semi-solid bead may be a liposomal bead. Solid beads may comprise metals including iron oxide, gold, and silver. In some cases, the bead may be a silica bead. In some cases, the bead can be rigid. In other cases, the bead may be flexible and/or compressible.

A bead may be of any suitable shape. Examples of bead shapes include, but are not limited to, spherical, non-spherical, oval, oblong, amorphous, circular, cylindrical, and variations thereof.

Beads may be of uniform size or heterogeneous size. In some cases, the diameter of a bead may be at least about 1 micrometers (μm), 5 μm, 10 μm, 20 μm, 30 μm, 40 μm, 50 μm, 60 μm, 70 μm, 80 μm, 90 μm, 100 μm, 250 μm, 500 μm, 1 mm, or greater. In some cases, a bead may have a diameter of less than about 5 μm, 10 μm, 20 μm, 30 μm, 40 μm, 50 μm, 60 μm, 70 μm, 80 μm, 90 μm, 100 μm, 250 μm, 500 μm, 1 mm, or less. In some cases, a bead may have a diameter in the range of about 40-75 μm, 30-75 μm, 20-75 μm, 40-85 μm, 40-95 μm, 20-100 μm, 10-100 μm, 1-100 μm, 20-250 μm, or 20-500 μm.

In certain aspects, beads can be provided as a population or plurality of beads having a relatively monodisperse size distribution. Where it may be desirable to provide relatively consistent amounts of reagents within partitions, maintaining relatively consistent bead characteristics, such as size, can contribute to the overall consistency. In particular, the beads described herein may have size distributions that have a coefficient of variation in their cross-sectional dimensions of less than 50%, less than 40%, less than 30%, less than 20%, and in some cases less than 15%, less than 10%, less than 5%, or less.

A bead may comprise natural and/or synthetic materials. For example, a bead can comprise a natural polymer, a synthetic polymer or both natural and synthetic polymers. Examples of natural polymers include proteins and sugars such as deoxyribonucleic acid, rubber, cellulose, carboxymethylcellulose, hydroxypropyl methylcellulose, starch (e.g., amylose, amylopectin), proteins, enzymes, polysaccharides, silks, polyhydroxyalkanoates, chitosan, dextran, collagen, carrageenan, ispaghula, acacia, agar, gelatin, shellac, sterculia gum, xanthan gum, Corn sugar gum, guar gum, gum karaya, agarose, alginic acid, alginate, or natural polymers thereof. Examples of synthetic polymers include acrylics, nylons, silicones, spandex, viscose rayon, polycarboxylic acids, polyvinyl acetate, polyacrylamide, polyacrylate, polyethylene glycol, polyurethanes, polylactic acid, silica, polystyrene, polyacrylonitrile, polybutadiene, polycarbonate, polyethylene, polyethylene terephthalate, poly(chlorotrifluoroethylene), poly(ethylene oxide), poly(ethylene terephthalate), polyethylene, polyisobutylene, poly(methyl methacrylate), poly(oxymethylene), polyformaldehyde, polypropylene, polystyrene, poly(tetrafluoroethylene), poly(vinyl acetate), poly(vinyl alcohol), poly(vinyl chloride), poly(vinylidene dichloride), poly(vinylidene difluoride), poly(vinyl fluoride) and/or combinations (e.g., co-polymers) thereof. Beads may also be formed from materials other than polymers, including lipids, micelles, ceramics, glass-ceramics, material composites, metals, other inorganic materials, and others.

In some instances, the bead may contain molecular precursors (e.g., monomers or polymers), which may form a polymer network via polymerization of the molecular precursors. In some cases, a precursor may be an already polymerized species capable of undergoing further polymerization via, for example, a chemical cross-linkage. In some cases, a precursor can comprise one or more of an acrylamide or a methacrylamide monomer, oligomer, or polymer. In some cases, the bead may comprise prepolymers, which are oligomers capable of further polymerization. For example, polyurethane beads may be prepared using prepolymers. In some cases, the bead may contain individual polymers that may be further polymerized together. In some cases, beads may be generated via polymerization of different precursors, such that they comprise mixed polymers, co-polymers, and/or block co-polymers. In some cases, the bead may comprise covalent or ionic bonds between polymeric precursors (e.g., monomers, oligomers, and linear polymers), nucleic acid molecules (e.g., oligonucleotides), primers, and other entities. In some cases, the covalent bonds can be carbon-carbon bonds or thioether bonds.

Cross-linking may be permanent or reversible, depending upon the particular cross-linker used. Reversible cross-linking may allow for the polymer to linearize or dissociate under appropriate conditions. In some cases, reversible cross-linking may also allow for reversible attachment of a material bound to the surface of a bead. In some cases, a cross-linker may form disulfide linkages. In some cases, the chemical cross-linker forming disulfide linkages may be cystamine or a modified cystamine.

In some cases, disulfide linkages can be formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, may be crosslinked with glutaraldehyde via hydrophilic chains to form a bead. Crosslinking of chitosan polymers may be achieved by chemical reactions that are initiated by heat, pressure, change in pH, and/or radiation.

In some cases, a bead may comprise an acrydite moiety, which in certain aspects may be used to attach one or more nucleic acid molecules (e.g., barcode sequence, barcoded nucleic acid molecule, barcoded oligonucleotide, primer, or other oligonucleotide) to the bead. In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., barcode sequence, barcoded nucleic acid molecule, barcoded oligonucleotide, primer, or other oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment can be reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., oligonucleotides) may be achieved through a wide range of different approaches, including activation of chemical groups within a polymer, incorporation of active or activatable functional groups in the polymer structure, or attachment at the pre-polymer or monomer stage in bead production.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., oligonucleotide), which may include a priming sequence (e.g., a primer for amplifying target nucleic acids, random primer, primer sequence for messenger RNA) and/or a one or more barcode sequences. The one more barcode sequences may include sequences that are the same for all nucleic acid molecules coupled to a given bead and/or sequences that are different across all nucleic acid molecules coupled to the given bead. The nucleic acid molecule may be incorporated into the bead.

In some cases, the nucleic acid molecule can comprise a functional sequence, for example, for attachment to a sequencing flow cell, such as, for example, a P5 sequence for Illumina® sequencing. In some cases, the nucleic acid molecule or derivative thereof (e.g., oligonucleotide or polynucleotide generated from the nucleic acid molecule) can comprise another functional sequence, such as, for example, a P7 sequence for attachment to a sequencing flow cell for Illumina sequencing. In some cases, the nucleic acid molecule can comprise a barcode sequence. In some cases, the primer can further comprise a unique molecular identifier (UMI). In some cases, the primer can comprise an R1 primer sequence for Illumina sequencing. In some cases, the primer can comprise an R2 primer sequence for Illumina sequencing. Examples of such nucleic acid molecules (e.g., oligonucleotides, polynucleotides, etc.) and uses thereof, as may be used with compositions, devices, methods and systems of the present disclosure, are provided in U.S. Patent Pub. Nos. 2014/0378345 and 2015/0376609, each of which is entirely incorporated herein by reference.

In some cases, precursors comprising a functional group that is reactive or capable of being activated such that it becomes reactive can be polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine can be co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NETS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions can be prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent:gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent may be used for reduction. Controlling the number of disulfide linkages that are reduced to free thiols may be useful in ensuring bead structural integrity during functionalization. In some cases, optically-active agents, such as fluorescent dyes may be coupled to beads via free thiol groups of the beads and used to quantify the number of free thiols present in a bead and/or track a bead.

In some cases, addition of moieties to a gel bead after gel bead formation may be advantageous. For example, addition of an oligonucleotide (e.g., barcoded oligonucleotide) after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

A bead injected or otherwise introduced into a partition may comprise releasably, cleavably, or reversibly attached barcodes. A bead injected or otherwise introduced into a partition may comprise activatable barcodes. A bead injected or otherwise introduced into a partition may be degradable, disruptable, or dissolvable beads.

Barcodes can be releasably, cleavably or reversibly attached to the beads such that barcodes can be released or be releasable through cleavage of a linkage between the barcode molecule and the bead, or released through degradation of the underlying bead itself, allowing the barcodes to be accessed or be accessible by other reagents, or both. In non-limiting examples, cleavage may be achieved through reduction of di-sulfide bonds, use of restriction enzymes, photo-activated cleavage, or cleavage via other types of stimuli (e.g., chemical, thermal, pH, enzymatic, etc.) and/or reactions, such as described elsewhere herein. Releasable barcodes may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to, or as an alternative to the cleavable linkages between the beads and the associated molecules, such as barcode containing nucleic acid molecules (e.g., barcoded oligonucleotides), the beads may be degradable, disruptable, or dissolvable spontaneously or upon exposure to one or more stimuli (e.g., temperature changes, pH changes, exposure to particular chemical species or phase, exposure to light, reducing agent, etc.). In some cases, a bead may be dissolvable, such that material components of the beads are solubilized when exposed to a particular chemical species or an environmental change, such as a change temperature or a change in pH. In some cases, a gel bead can be degraded or dissolved at elevated temperature and/or in basic conditions. In some cases, a bead may be thermally degradable such that when the bead is exposed to an appropriate change in temperature (e.g., heat), the bead degrades. Degradation or dissolution of a bead bound to a species (e.g., a nucleic acid molecule, e.g., barcoded oligonucleotide) may result in release of the species from the bead.

As will be appreciated from the above disclosure, the degradation of a bead may refer to the disassociation of a bound or entrained species from a bead, both with and without structurally degrading the physical bead itself. For example, the degradation of the bead may involve cleavage of a cleavable linkage via one or more species and/or methods described elsewhere herein. In another example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

A degradable bead may be introduced into a partition, such as a droplet of an emulsion or a well, such that the bead degrades within the partition and any associated species (e.g., oligonucleotides) are released within the droplet when the appropriate stimulus is applied. The free species (e.g., oligonucleotides, nucleic acid molecules) may interact with other reagents contained in the partition. For example, a polyacrylamide bead comprising cystamine and linked, via a disulfide bond, to a barcode sequence, may be combined with a reducing agent within a droplet of a water-in-oil emulsion. Within the droplet, the reducing agent can break the various disulfide bonds, resulting in bead degradation and release of the barcode sequence into the aqueous, inner environment of the droplet. In another example, heating of a droplet comprising a bead-bound barcode sequence in basic solution may also result in bead degradation and release of the attached barcode sequence into the aqueous, inner environment of the droplet.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing nucleic acid molecule (e.g., oligonucleotide) bearing beads.

In some cases, beads can be non-covalently loaded with one or more reagents. The beads can be non-covalently loaded by, for instance, subjecting the beads to conditions sufficient to swell the beads, allowing sufficient time for the reagents to diffuse into the interiors of the beads, and subjecting the beads to conditions sufficient to de-swell the beads. The swelling of the beads may be accomplished, for instance, by placing the beads in a thermodynamically favorable solvent, subjecting the beads to a higher or lower temperature, subjecting the beads to a higher or lower ion concentration, and/or subjecting the beads to an electric field. The swelling of the beads may be accomplished by various swelling methods. The de-swelling of the beads may be accomplished, for instance, by transferring the beads in a thermodynamically unfavorable solvent, subjecting the beads to lower or high temperatures, subjecting the beads to a lower or higher ion concentration, and/or removing an electric field. The de-swelling of the beads may be accomplished by various de-swelling methods. Transferring the beads may cause pores in the bead to shrink. The shrinking may then hinder reagents within the beads from diffusing out of the interiors of the beads. The hindrance may be due to steric interactions between the reagents and the interiors of the beads. The transfer may be accomplished microfluidically. For instance, the transfer may be achieved by moving the beads from one co-flowing solvent stream to a different co-flowing solvent stream. The swellability and/or pore size of the beads may be adjusted by changing the polymer composition of the bead.

In some cases, an acrydite moiety linked to a precursor, another species linked to a precursor, or a precursor itself can comprise a labile bond, such as chemically, thermally, or photo-sensitive bond e.g., disulfide bond, UV sensitive bond, or the like. Once acrydite moieties or other moieties comprising a labile bond are incorporated into a bead, the bead may also comprise the labile bond. The labile bond may be, for example, useful in reversibly linking (e.g., covalently linking) species (e.g., barcodes, primers, etc.) to a bead. In some cases, a thermally labile bond may include a nucleic acid hybridization based attachment, e.g., where an oligonucleotide is hybridized to a complementary sequence that is attached to the bead, such that thermal melting of the hybrid releases the oligonucleotide, e.g., a barcode containing sequence, from the bead or microcapsule.

The addition of multiple types of labile bonds to a gel bead may result in the generation of a bead capable of responding to varied stimuli. Each type of labile bond may be sensitive to an associated stimulus (e.g., chemical stimulus, light, temperature, enzymatic, etc.) such that release of species attached to a bead via each labile bond may be controlled by the application of the appropriate stimulus. Such functionality may be useful in controlled release of species from a gel bead. In some cases, another species comprising a labile bond may be linked to a gel bead after gel bead formation via, for example, an activated functional group of the gel bead as described above. As will be appreciated, barcodes that are releasably, cleavably or reversibly attached to the beads described herein include barcodes that are released or releasable through cleavage of a linkage between the barcode molecule and the bead, or that are released through degradation of the underlying bead itself, allowing the barcodes to be accessed or accessible by other reagents, or both.

The barcodes that are releasable as described herein may sometimes be referred to as being activatable, in that they are available for reaction once released. Thus, for example, an activatable barcode may be activated by releasing the barcode from a bead (or other suitable type of partition described herein). Other activatable configurations are also envisioned in the context of the described methods and systems.

In addition to thermally cleavable bonds, disulfide bonds and UV sensitive bonds, other non-limiting examples of labile bonds that may be coupled to a precursor or bead include an ester linkage (e.g., cleavable with an acid, a base, or hydroxylamine), a vicinal diol linkage (e.g., cleavable via sodium periodate), a Diels-Alder linkage (e.g., cleavable via heat), a sulfone linkage (e.g., cleavable via a base), a silyl ether linkage (e.g., cleavable via an acid), a glycosidic linkage (e.g., cleavable via an amylase), a peptide linkage (e.g., cleavable via a protease), or a phosphodiester linkage (e.g., cleavable via a nuclease (e.g., DNAase)). A bond may be cleavable via other nucleic acid molecule targeting enzymes, such as restriction enzymes (e.g., restriction endonucleases), as described further below.

Species may be encapsulated in beads during bead generation (e.g., during polymerization of precursors). Such species may or may not participate in polymerization. Such species may be entered into polymerization reaction mixtures such that generated beads comprise the species upon bead formation. In some cases, such species may be added to the gel beads after formation. Such species may include, for example, nucleic acid molecules (e.g., oligonucleotides), reagents for a nucleic acid amplification reaction (e.g., primers, polymerases, dNTPs, co-factors (e.g., ionic co-factors), buffers) including those described herein, reagents for enzymatic reactions (e.g., enzymes, co-factors, substrates, buffers), reagents for nucleic acid modification reactions such as polymerization, ligation, or digestion, and/or reagents for template preparation (e.g., tagmentation) for one or more sequencing platforms (e.g., Nextera® for Illumina®). Such species may include one or more enzymes described herein, including without limitation, polymerase, reverse transcriptase, restriction enzymes (e.g., endonuclease), transposase, ligase, proteinase K, DNAse, etc. Such species may include one or more reagents described elsewhere herein (e.g., lysis agents, inhibitors, inactivating agents, chelating agents, stimulus). Trapping of such species may be controlled by the polymer network density generated during polymerization of precursors, control of ionic charge within the gel bead (e.g., via ionic species linked to polymerized species), or by the release of other species. Encapsulated species may be released from a bead upon bead degradation and/or by application of a stimulus capable of releasing the species from the bead. Alternatively or in addition, species may be partitioned in a partition (e.g., droplet) during or subsequent to partition formation. Such species may include, without limitation, the abovementioned species that may also be encapsulated in a bead.

A degradable bead may comprise one or more species with a labile bond such that, when the bead/species is exposed to the appropriate stimuli, the bond is broken and the bead degrades. The labile bond may be a chemical bond (e.g., covalent bond, ionic bond) or may be another type of physical interaction (e.g., van der Waals interactions, dipole-dipole interactions, etc.). In some cases, a crosslinker used to generate a bead may comprise a labile bond. Upon exposure to the appropriate conditions, the labile bond can be broken and the bead degraded. For example, upon exposure of a polyacrylamide gel bead comprising cystamine crosslinkers to a reducing agent, the disulfide bonds of the cystamine can be broken and the bead degraded.

A degradable bead may be useful in more quickly releasing an attached species (e.g., a nucleic acid molecule, a barcode sequence, a primer, etc) from the bead when the appropriate stimulus is applied to the bead as compared to a bead that does not degrade. For example, for a species bound to an inner surface of a porous bead or in the case of an encapsulated species, the species may have greater mobility and accessibility to other species in solution upon degradation of the bead. In some cases, a species may also be attached to a degradable bead via a degradable linker (e.g., disulfide linker). The degradable linker may respond to the same stimuli as the degradable bead or the two degradable species may respond to different stimuli. For example, a barcode sequence may be attached, via a disulfide bond, to a polyacrylamide bead comprising cystamine. Upon exposure of the barcoded-bead to a reducing agent, the bead degrades and the barcode sequence is released upon breakage of both the disulfide linkage between the barcode sequence and the bead and the disulfide linkages of the cystamine in the bead.

As will be appreciated from the above disclosure, while referred to as degradation of a bead, in many instances as noted above, that degradation may refer to the disassociation of a bound or entrained species from a bead, both with and without structurally degrading the physical bead itself. For example, entrained species may be released from beads through osmotic pressure differences due to, for example, changing chemical environments. By way of example, alteration of bead pore sizes due to osmotic pressure differences can generally occur without structural degradation of the bead itself. In some cases, an increase in pore size due to osmotic swelling of a bead can permit the release of entrained species within the bead. In other cases, osmotic shrinking of a bead may cause a bead to better retain an entrained species due to pore size contraction.

Where degradable beads are provided, it may be beneficial to avoid exposing such beads to the stimulus or stimuli that cause such degradation prior to a given time, in order to, for example, avoid premature bead degradation and issues that arise from such degradation, including for example poor flow characteristics and aggregation. By way of example, where beads comprise reducible cross-linking groups, such as disulfide groups, it will be desirable to avoid contacting such beads with reducing agents, e.g., DTT or other disulfide cleaving reagents. In such cases, treatment to the beads described herein will, in some cases be provided free of reducing agents, such as DTT. Because reducing agents are often provided in commercial enzyme preparations, it may be desirable to provide reducing agent free (or DTT free) enzyme preparations in treating the beads described herein. Examples of such enzymes include, e.g., polymerase enzyme preparations, reverse transcriptase enzyme preparations, ligase enzyme preparations, as well as many other enzyme preparations that may be used to treat the beads described herein. The terms “reducing agent free” or “DTT free” preparations can refer to a preparation having less than about 1/10th, less than about 1/50th, or even less than about 1/100th of the lower ranges for such materials used in degrading the beads. For example, for DTT, the reducing agent free preparation can have less than about 0.01 millimolar (mM), 0.005 mM, 0.001 mM DTT, 0.0005 mM DTT, or even less than about 0.0001 mM DTT. In many cases, the amount of DTT can be undetectable.

Numerous chemical triggers may be used to trigger the degradation of beads. Examples of these chemical changes may include, but are not limited to pH-mediated changes to the integrity of a component within the bead, degradation of a component of a bead via cleavage of cross-linked bonds, and depolymerization of a component of a bead.

In some embodiments, a bead may be formed from materials that comprise degradable chemical crosslinkers, such as BAC or cystamine. Degradation of such degradable crosslinkers may be accomplished through a number of mechanisms. In some examples, a bead may be contacted with a chemical degrading agent that may induce oxidation, reduction or other chemical changes. For example, a chemical degrading agent may be a reducing agent, such as dithiothreitol (DTT). Additional examples of reducing agents may include β-mercaptoethanol, (2S)-2-amino-1,4-dimercaptobutane (dithiobutylamine or DTBA), tris(2-carboxyethyl) phosphine (TCEP), or combinations thereof. A reducing agent may degrade the disulfide bonds formed between gel precursors forming the bead, and thus, degrade the bead. In other cases, a change in pH of a solution, such as an increase in pH, may trigger degradation of a bead. In other cases, exposure to an aqueous solution, such as water, may trigger hydrolytic degradation, and thus degradation of the bead.

Beads may also be induced to release their contents upon the application of a thermal stimulus. A change in temperature can cause a variety of changes to a bead. For example, heat can cause a solid bead to liquefy. A change in heat may cause melting of a bead such that a portion of the bead degrades. In other cases, heat may increase the internal pressure of the bead components such that the bead ruptures or explodes. Heat may also act upon heat-sensitive polymers used as materials to construct beads.

Any suitable agent may degrade beads. In some embodiments, changes in temperature or pH may be used to degrade thermo-sensitive or pH-sensitive bonds within beads. In some embodiments, chemical degrading agents may be used to degrade chemical bonds within beads by oxidation, reduction or other chemical changes. For example, a chemical degrading agent may be a reducing agent, such as DTT, wherein DTT may degrade the disulfide bonds formed between a crosslinker and gel precursors, thus degrading the bead. In some embodiments, a reducing agent may be added to degrade the bead, which may or may not cause the bead to release its contents. Examples of reducing agents may include dithiothreitol (DTT), β-mercaptoethanol, (2S)-2-amino-1,4-dimercaptobutane (dithiobutylamine or DTBA), tris(2-carboxyethyl) phosphine (TCEP), or combinations thereof. The reducing agent may be present at a concentration of about 0.1 mM, 0.5 mM, 1 mM, 5 mM, 10 mM. The reducing agent may be present at a concentration of at least about 0.1 mM, 0.5 mM, 1 mM, 5 mM, 10 mM, or greater than 10 mM. The reducing agent may be present at concentration of at most about 10 mM, 5 mM, 1 mM, 0.5 mM, 0.1 mM, or less.

Any suitable number of molecular tag molecules (e.g., primer, barcoded oligonucleotide) can be associated with a bead such that, upon release from the bead, the molecular tag molecules (e.g., primer, e.g., barcoded oligonucleotide) are present in the partition at a pre-defined concentration. Such pre-defined concentration may be selected to facilitate certain reactions for generating a sequencing library, e.g., amplification, within the partition. In some cases, the pre-defined concentration of the primer can be limited by the process of producing oligonucleotide bearing beads.

Although FIG. 1 and FIG. 2 have been described in terms of providing substantially singly occupied partitions, above, in certain cases, it may be desirable to provide multiply occupied partitions, e.g., containing two, three, four or more cells and/or microcapsules (e.g., beads) comprising barcoded nucleic acid molecules (e.g., oligonucleotides) within a single partition. Accordingly, as noted above, the flow characteristics of the biological particle and/or bead containing fluids and partitioning fluids may be controlled to provide for such multiply occupied partitions. In particular, the flow parameters may be controlled to provide a given occupancy rate at greater than about 50% of the partitions, greater than about 75%, and in some cases greater than about 80%, 90%, 95%, or higher.

In some cases, additional microcapsules can be used to deliver additional reagents to a partition. In such cases, it may be advantageous to introduce different beads into a common channel or droplet generation junction, from different bead sources (e.g., containing different associated reagents) through different channel inlets into such common channel or droplet generation junction (e.g., junction 210). In such cases, the flow and frequency of the different beads into the channel or junction may be controlled to provide for a certain ratio of microcapsules from each source, while ensuring a given pairing or combination of such beads into a partition with a given number of biological particles (e.g., one biological particle and one bead per partition).

The partitions described herein may comprise small volumes, for example, less than about 10 microliters (μL), 5 μL, 1 μL, 900 picoliters (pL), 800 pL, 700 pL, 600 pL, 500 pL, 400 pL, 300 pL, 200 pL, 100 pL, 50 pL, 20 pL, 10 pL, 1 pL, 500 nanoliters (nL), 100 nL, 50 nL, or less.

For example, in the case of droplet based partitions, the droplets may have overall volumes that are less than about 1000 pL, 900 pL, 800 pL, 700 pL, 600 pL, 500 pL, 400 pL, 300 pL, 200 pL, 100 pL, 50 pL, 20 pL, 10 pL, 1 pL, or less. Where co-partitioned with microcapsules, it will be appreciated that the sample fluid volume, e.g., including co-partitioned biological particles and/or beads, within the partitions may be less than about 90% of the above described volumes, less than about 80%, less than about 70%, less than about 60%, less than about 50%, less than about 40%, less than about 30%, less than about 20%, or less than about 10% of the above described volumes.

As is described elsewhere herein, partitioning species may generate a population or plurality of partitions. In such cases, any suitable number of partitions can be generated or otherwise provided. For example, at least about 1,000 partitions, at least about 5,000 partitions, at least about 10,000 partitions, at least about 50,000 partitions, at least about 100,000 partitions, at least about 500,000 partitions, at least about 1,000,000 partitions, at least about 5,000,000 partitions at least about 10,000,000 partitions, at least about 50,000,000 partitions, at least about 100,000,000 partitions, at least about 500,000,000 partitions, at least about 1,000,000,000 partitions, or more partitions can be generated or otherwise provided. Moreover, the plurality of partitions may comprise both unoccupied partitions (e.g., empty partitions) and occupied partitions.

### Reagents

In accordance with certain aspects, biological particles may be partitioned along with lysis reagents in order to release the contents of the biological particles within the partition. In such cases, the lysis agents can be contacted with the biological particle suspension concurrently with, or immediately prior to, the introduction of the biological particles into the partitioning junction/droplet generation zone (e.g., junction 210), such as through an additional channel or channels upstream of the channel junction. In accordance with other aspects, additionally or alternatively, biological particles may be partitioned along with other reagents, as will be described further below.

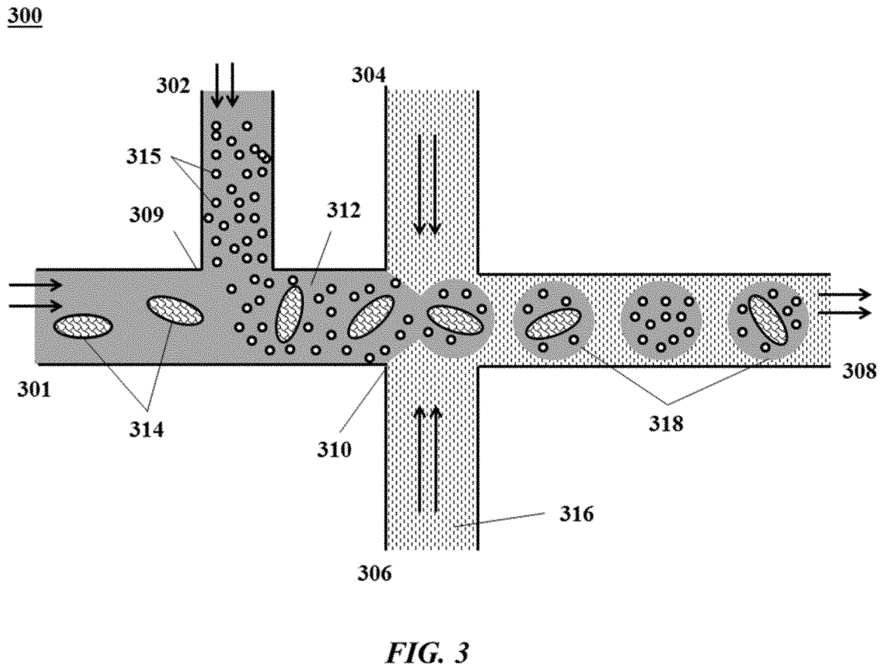

FIG. 3 shows an example of a microfluidic channel structure 300 for co-partitioning biological particles and reagents. The channel structure 300 can include channel segments 301, 302, 304, 306 and 308. Channel segments 301 and 302 communicate at a first channel junction 309. Channel segments 302, 304, 306, and 308 communicate at a second channel junction 310.

In an example operation, the channel segment 301 may transport an aqueous fluid 312 that includes a plurality of biological particles 314 along the channel segment 301 into the second junction 310. As an alternative or in addition to, channel segment 301 may transport beads (e.g., gel beads). The beads may comprise barcode molecules.

For example, the channel segment 301 may be connected to a reservoir comprising an aqueous suspension of biological particles 314. Upstream of, and immediately prior to reaching, the second junction 310, the channel segment 301 may meet the channel segment 302 at the first junction 309. The channel segment 302 may transport a plurality of reagents 315 (e.g., lysis agents) suspended in the aqueous fluid 312 along the channel segment 302 into the first junction 309. For example, the channel segment 302 may be connected to a reservoir comprising the reagents 315. After the first junction 309, the aqueous fluid 312 in the channel segment 301 can carry both the biological particles 314 and the reagents 315 towards the second junction 310. In some instances, the aqueous fluid 312 in the channel segment 301 can include one or more reagents, which can be the same or different reagents as the reagents 315. A second fluid 316 that is immiscible with the aqueous fluid 312 (e.g., oil) can be delivered to the second junction 310 from each of channel segments 304 and 306. Upon meeting of the aqueous fluid 312 from the channel segment 301 and the second fluid 316 from each of channel segments 304 and 306 at the second channel junction 310, the aqueous fluid 312 can be partitioned as discrete droplets 318 in the second fluid 316 and flow away from the second junction 310 along channel segment 308. The channel segment 308 may deliver the discrete droplets 318 to an outlet reservoir fluidly coupled to the channel segment 308, where they may be harvested.

The second fluid 316 can comprise an oil, such as a fluorinated oil, that includes a fluorosurfactant for stabilizing the resulting droplets, for example, inhibiting subsequent coalescence of the resulting droplets 318.

A discrete droplet generated may include an individual biological particle 314 and/or one or more reagents 315. In some instances, a discrete droplet generated may include a barcode carrying bead (not shown), such as via other microfluidics structures described elsewhere herein. In some instances, a discrete droplet may be unoccupied (e.g., no reagents, no biological particles).

Beneficially, when lysis reagents and biological particles are co-partitioned, the lysis reagents can facilitate the release of the contents of the biological particles within the partition. The contents released in a partition may remain discrete from the contents of other partitions.

As will be appreciated, the channel segments described herein may be coupled to any of a variety of different fluid sources or receiving components, including reservoirs, tubing, manifolds, or fluidic components of other systems. As will be appreciated, the microfluidic channel structure 300 may have other geometries. For example, a microfluidic channel structure can have more than two channel junctions. For example, a microfluidic channel structure can have 2, 3, 4, 5 channel segments or more each carrying the same or different types of beads, reagents, and/or biological particles that meet at a channel junction. Fluid flow in each channel segment may be controlled to control the partitioning of the different elements into droplets. Fluid may be directed flow along one or more channels or reservoirs via one or more fluid flow units. A fluid flow unit can comprise compressors (e.g., providing positive pressure), pumps (e.g., providing negative pressure), actuators, and the like to control flow of the fluid. Fluid may also or otherwise be controlled via applied pressure differentials, centrifugal force, electrokinetic pumping, vacuum, capillary or gravity flow, or the like.

Examples of lysis agents include bioactive reagents, such as lysis enzymes that are used for lysis of different cell types, e.g., gram positive or negative bacteria, plants, yeast, mammalian, etc., such as lysozymes, achromopeptidase, lysostaphin, labiase, kitalase, lyticase, and a variety of other lysis enzymes available from, e.g., Sigma-Aldrich, Inc. (St Louis, Mo.), as well as other commercially available lysis enzymes. Other lysis agents may additionally or alternatively be co-partitioned with the biological particles to cause the release of the biological particles's contents into the partitions. For example, in some cases, surfactant-based lysis solutions may be used to lyse cells, although these may be less desirable for emulsion based systems where the surfactants can interfere with stable emulsions. In some cases, lysis solutions may include non-ionic surfactants such as, for example, TritonX-100 and Tween 20. In some cases, lysis solutions may include ionic surfactants such as, for example, sarcosyl and sodium dodecyl sulfate (SDS). Electroporation, thermal, acoustic or mechanical cellular disruption may also be used in certain cases, e.g., non-emulsion based partitioning such as encapsulation of biological particles that may be in addition to or in place of droplet partitioning, where any pore size of the encapsulate is sufficiently small to retain nucleic acid fragments of a given size, following cellular disruption.

In addition to the lysis agents co-partitioned with the biological particles described above, other reagents can also be co-partitioned with the biological particles, including, for example, DNase and RNase inactivating agents or inhibitors, such as proteinase K, chelating agents, such as EDTA, and other reagents employed in removing or otherwise reducing negative activity or impact of different cell lysate components on subsequent processing of nucleic acids. In addition, in the case of encapsulated biological particles, the biological particles may be exposed to an appropriate stimulus to release the biological particles or their contents from a co-partitioned microcapsule. For example, in some cases, a chemical stimulus may be co-partitioned along with an encapsulated biological particle to allow for the degradation of the microcapsule and release of the cell or its contents into the larger partition. In some cases, this stimulus may be the same as the stimulus described elsewhere herein for release of nucleic acid molecules (e.g., oligonucleotides) from their respective microcapsule (e.g., bead). In alternative aspects, this may be a different and non-overlapping stimulus, in order to allow an encapsulated biological particle to be released into a partition at a different time from the release of nucleic acid molecules into the same partition.

Additional reagents may also be co-partitioned with the biological particles, such as endonucleases to fragment a biological particle's DNA, DNA polymerase enzymes and dNTPs used to amplify the biological particle's nucleic acid fragments and to attach the barcode molecular tags to the amplified fragments. Other enzymes may be co-partitioned, including without limitation, polymerase, transposase, ligase, proteinase K, DNAse, etc. Additional reagents may also include reverse transcriptase enzymes, including enzymes with terminal transferase activity, primers and oligonucleotides, and switch oligonucleotides (also referred to herein as “switch oligos” or “template switching oligonucleotides”) which can be used for template switching. In some cases, template switching can be used to increase the length of a cDNA. In some cases, template switching can be used to append a predefined nucleic acid sequence to the cDNA. In an example of template switching, cDNA can be generated from reverse transcription of a template, e.g., cellular mRNA, where a reverse transcriptase with terminal transferase activity can add additional nucleotides, e.g., polyC, to the cDNA in a template independent manner. Switch oligos can include sequences complementary to the additional nucleotides, e.g., polyG. The additional nucleotides (e.g., polyC) on the cDNA can hybridize to the additional nucleotides (e.g., polyG) on the switch oligo, whereby the switch oligo can be used by the reverse transcriptase as template to further extend the cDNA. Template switching oligonucleotides may comprise a hybridization region and a template region. The hybridization region can comprise any sequence capable of hybridizing to the target. In some cases, as previously described, the hybridization region comprises a series of G bases to complement the overhanging C bases at the 3′ end of a cDNA molecule. The series of G bases may comprise 1 G base, 2 G bases, 3 G bases, 4 G bases, 5 G bases or more than 5 G bases. The template sequence can comprise any sequence to be incorporated into the cDNA. In some cases, the template region comprises at least 1 (e.g., at least 2, 3, 4, 5 or more) tag sequences and/or functional sequences. Switch oligos may comprise deoxyribonucleic acids; ribonucleic acids; modified nucleic acids including 2-Aminopurine, 2,6-Diaminopurine (2-Amino-dA), inverted dT, 5-Methyl dC, 2′-deoxyInosine, Super T (5-hydroxybutynl-2′-deoxyuridine), Super G (8-aza-7-deazaguanosine), locked nucleic acids (LNAs), unlocked nucleic acids (UNAs, e.g., UNA-A, UNA-U, UNA-C, UNA-G), Iso-dG, Iso-dC, 2′ Fluoro bases (e.g., Fluoro C, Fluoro U, Fluoro A, and Fluoro G), or any combination.

In some cases, the length of a switch oligo may be at least about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides or longer.

In some cases, the length of a switch oligo may be at most about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249 or 250 nucleotides.

Once the contents of the cells are released into their respective partitions, the macromolecular components (e.g., macromolecular constituents of biological particles, such as RNA, DNA, or proteins) contained therein may be further processed within the partitions. In accordance with the methods and systems described herein, the macromolecular component contents of individual biological particles can be provided with unique identifiers such that, upon characterization of those macromolecular components they may be attributed as having been derived from the same biological particle or particles. The ability to attribute characteristics to individual biological particles or groups of biological particles is provided by the assignment of unique identifiers specifically to an individual biological particle or groups of biological particles. Unique identifiers, e.g., in the form of nucleic acid barcodes can be assigned or associated with individual biological particles or populations of biological particle, in order to tag or label the biological particle's macromolecular components (and as a result, its characteristics) with the unique identifiers. These unique identifiers can then be used to attribute the biological particle's components and characteristics to an individual biological particle or group of biological particles.

In some aspects, this is performed by co-partitioning the individual biological particle or groups of biological particles with the unique identifiers, such as described above (with reference to FIG. 2). In some aspects, the unique identifiers are provided in the form of nucleic acid molecules (e.g., oligonucleotides) that comprise nucleic acid barcode sequences that may be attached to or otherwise associated with the nucleic acid contents of individual biological particle, or to other components of the biological particle, and particularly to fragments of those nucleic acids. The nucleic acid molecules are partitioned such that as between nucleic acid molecules in a given partition, the nucleic acid barcode sequences contained therein are the same, but as between different partitions, the nucleic acid molecule can, and do have differing barcode sequences, or at least represent a large number of different barcode sequences across all of the partitions in a given analysis. In some aspects, only one nucleic acid barcode sequence can be associated with a given partition, although in some cases, two or more different barcode sequences may be present.

The nucleic acid barcode sequences can include from about 6 to about 20 or more nucleotides within the sequence of the nucleic acid molecules (e.g., oligonucleotides). In some cases, the length of a barcode sequence may be about 6, 7, 8, 9, 10, 11, 12, 13, 14, 15, 16, 17, 18, 19, 20 nucleotides or longer. In some cases, the length of a barcode sequence may be at least about 6, 7, 8, 9, 10, 11, 12, 13, 14, 15, 16, 17, 18, 19, 20 nucleotides or longer. In some cases, the length of a barcode sequence may be at most about 6, 7, 8, 9, 10, 11, 12, 13, 14, 15, 16, 17, 18, 19, 20 nucleotides or shorter. These nucleotides may be completely contiguous, i.e., in a single stretch of adjacent nucleotides, or they may be separated into two or more separate subsequences that are separated by 1 or more nucleotides. In some cases, separated barcode subsequences can be from about 4 to about 16 nucleotides in length. In some cases, the barcode subsequence may be about 4, 5, 6, 7, 8, 9, 10, 11, 12, 13, 14, 15, 16 nucleotides or longer. In some cases, the barcode subsequence may be at least about 4, 5, 6, 7, 8, 9, 10, 11, 12, 13, 14, 15, 16 nucleotides or longer. In some cases, the barcode subsequence may be at most about 4, 5, 6, 7, 8, 9, 10, 11, 12, 13, 14, 15, 16 nucleotides or shorter.

The co-partitioned nucleic acid molecules can also comprise other functional sequences useful in the processing of the nucleic acids from the co-partitioned biological particles. These sequences include, e.g., targeted or random/universal amplification primer sequences for amplifying the genomic DNA from the individual biological particles within the partitions while attaching the associated barcode sequences, sequencing primers or primer recognition sites, hybridization or probing sequences, e.g., for identification of presence of the sequences or for pulling down barcoded nucleic acids, or any of a number of other potential functional sequences. Other mechanisms of co-partitioning oligonucleotides may also be employed, including, e.g., coalescence of two or more droplets, where one droplet contains oligonucleotides, or microdispensing of oligonucleotides into partitions, e.g., droplets within microfluidic systems.

In an example, microcapsules, such as beads, are provided that each include large numbers of the above described barcoded nucleic acid molecules (e.g., barcoded oligonucleotides) releasably attached to the beads, where all of the nucleic acid molecules attached to a particular bead will include the same nucleic acid barcode sequence, but where a large number of diverse barcode sequences are represented across the population of beads used. In some embodiments, hydrogel beads, e.g., comprising polyacrylamide polymer matrices, are used as a solid support and delivery vehicle for the nucleic acid molecules into the partitions, as they are capable of carrying large numbers of nucleic acid molecules, and may be configured to release those nucleic acid molecules upon exposure to a particular stimulus, as described elsewhere herein. In some cases, the population of beads provides a diverse barcode sequence library that includes at least about 1,000 different barcode sequences, at least about 5,000 different barcode sequences, at least about 10,000 different barcode sequences, at least about 50,000 different barcode sequences, at least about 100,000 different barcode sequences, at least about 1,000,000 different barcode sequences, at least about 5,000,000 different barcode sequences, or at least about 10,000,000 different barcode sequences, or more. Additionally, each bead can be provided with large numbers of nucleic acid (e.g., oligonucleotide) molecules attached. In particular, the number of molecules of nucleic acid molecules including the barcode sequence on an individual bead can be at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules, or more. Nucleic acid molecules of a given bead can include identical (or common) barcode sequences, different barcode sequences, or a combination of both. Nucleic acid molecules of a given bead can include multiple sets of nucleic acid molecules. Nucleic acid molecules of a given set can include identical barcode sequences. The identical barcode sequences can be different from barcode sequences of nucleic acid molecules of another set.

Moreover, when the population of beads is partitioned, the resulting population of partitions can also include a diverse barcode library that includes at least about 1,000 different barcode sequences, at least about 5,000 different barcode sequences, at least about 10,000 different barcode sequences, at least at least about 50,000 different barcode sequences, at least about 100,000 different barcode sequences, at least about 1,000,000 different barcode sequences, at least about 5,000,000 different barcode sequences, or at least about 10,000,000 different barcode sequences. Additionally, each partition of the population can include at least about 1,000 nucleic acid molecules, at least about 5,000 nucleic acid molecules, at least about 10,000 nucleic acid molecules, at least about 50,000 nucleic acid molecules, at least about 100,000 nucleic acid molecules, at least about 500,000 nucleic acids, at least about 1,000,000 nucleic acid molecules, at least about 5,000,000 nucleic acid molecules, at least about 10,000,000 nucleic acid molecules, at least about 50,000,000 nucleic acid molecules, at least about 100,000,000 nucleic acid molecules, at least about 250,000,000 nucleic acid molecules and in some cases at least about 1 billion nucleic acid molecules.

In some cases, it may be desirable to incorporate multiple different barcodes within a given partition, either attached to a single or multiple beads within the partition. For example, in some cases, a mixed, but known set of barcode sequences may provide greater assurance of identification in the subsequent processing, e.g., by providing a stronger address or attribution of the barcodes to a given partition, as a duplicate or independent confirmation of the output from a given partition.

The nucleic acid molecules (e.g., oligonucleotides) are releasable from the beads upon the application of a particular stimulus to the beads. In some cases, the stimulus may be a photo-stimulus, e.g., through cleavage of a photo-labile linkage that releases the nucleic acid molecules. In other cases, a thermal stimulus may be used, where elevation of the temperature of the beads environment will result in cleavage of a linkage or other release of the nucleic acid molecules form the beads. In still other cases, a chemical stimulus can be used that cleaves a linkage of the nucleic acid molecules to the beads, or otherwise results in release of the nucleic acid molecules from the beads. In one case, such compositions include the polyacrylamide matrices described above for encapsulation of biological particles, and may be degraded for release of the attached nucleic acid molecules through exposure to a reducing agent, such as DTT.

In some aspects, provided are systems and methods for controlled partitioning. Droplet size may be controlled by adjusting certain geometric features in channel architecture (e.g., microfluidics channel architecture). For example, an expansion angle, width, and/or length of a channel may be adjusted to control droplet size.

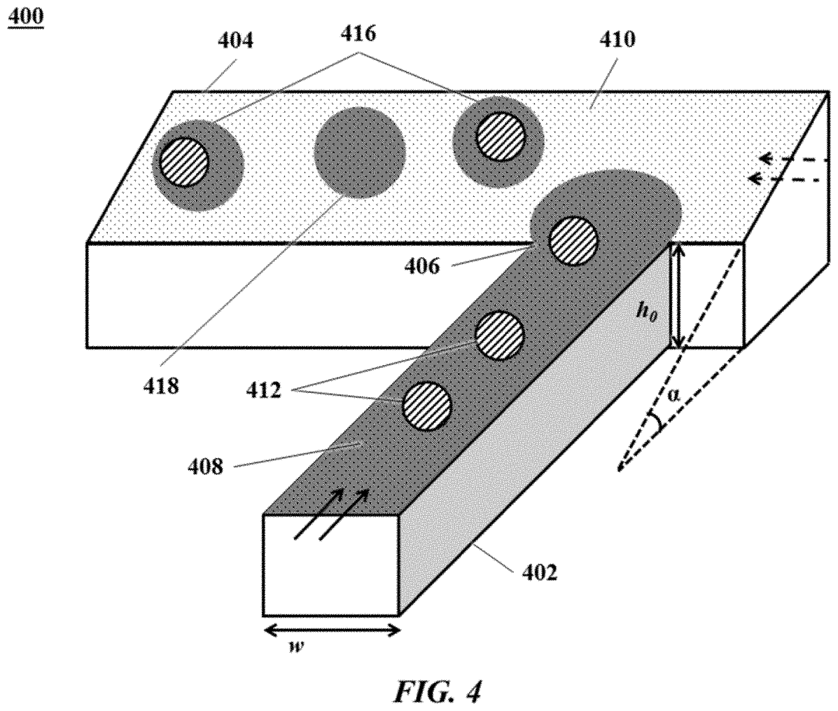

FIG. 4 shows an example of a microfluidic channel structure for the controlled partitioning of beads into discrete droplets. A channel structure 400 can include a channel segment 402 communicating at a channel junction 406 (or intersection) with a reservoir 404. The reservoir 404 can be a chamber. Any reference to “reservoir,” as used herein, can also refer to a “chamber.” In operation, an aqueous fluid 408 that includes suspended beads 412 may be transported along the channel segment 402 into the junction 406 to meet a second fluid 410 that is immiscible with the aqueous fluid 408 in the reservoir 404 to create droplets 416, 418 of the aqueous fluid 408 flowing into the reservoir 404. At the juncture 406 where the aqueous fluid 408 and the second fluid 410 meet, droplets can form based on factors such as the hydrodynamic forces at the juncture 406, flow rates of the two fluids 408, 410, fluid properties, and certain geometric parameters (e.g., w, h₀, α, etc.) of the channel structure 400. A plurality of droplets can be collected in the reservoir 404 by continuously injecting the aqueous fluid 408 from the channel segment 402 through the juncture 406.

A discrete droplet generated may include a bead (e.g., as in occupied droplets 416). Alternatively, a discrete droplet generated may include more than one bead. Alternatively, a discrete droplet generated may not include any beads (e.g., as in unoccupied droplet 418). In some instances, a discrete droplet generated may contain one or more biological particles, as described elsewhere herein. In some instances, a discrete droplet generated may comprise one or more reagents, as described elsewhere herein.

In some instances, the aqueous fluid 408 can have a substantially uniform concentration or frequency of beads 412. The beads 412 can be introduced into the channel segment 402 from a separate channel (not shown in FIG. 4). The frequency of beads 412 in the channel segment 402 may be controlled by controlling the frequency in which the beads 412 are introduced into the channel segment 402 and/or the relative flow rates of the fluids in the channel segment 402 and the separate channel. In some instances, the beads can be introduced into the channel segment 402 from a plurality of different channels, and the frequency controlled accordingly.

In some instances, the aqueous fluid 408 in the channel segment 402 can comprise biological particles (e.g., described with reference to FIGS. 1 and 2). In some instances, the aqueous fluid 408 can have a substantially uniform concentration or frequency of biological particles. As with the beads, the biological particles can be introduced into the channel segment 402 from a separate channel. The frequency or concentration of the biological particles in the aqueous fluid 408 in the channel segment 402 may be controlled by controlling the frequency in which the biological particles are introduced into the channel segment 402 and/or the relative flow rates of the fluids in the channel segment 402 and the separate channel. In some instances, the biological particles can be introduced into the channel segment 402 from a plurality of different channels, and the frequency controlled accordingly. In some instances, a first separate channel can introduce beads and a second separate channel can introduce biological particles into the channel segment 402. The first separate channel introducing the beads may be upstream or downstream of the second separate channel introducing the biological particles.

The second fluid 410 can comprise an oil, such as a fluorinated oil, that includes a fluorosurfactant for stabilizing the resulting droplets, for example, inhibiting subsequent coalescence of the resulting droplets.

In some instances, the second fluid 410 may not be subjected to and/or directed to any flow in or out of the reservoir 404. For example, the second fluid 410 may be substantially stationary in the reservoir 404. In some instances, the second fluid 410 may be subjected to flow within the reservoir 404, but not in or out of the reservoir 404, such as via application of pressure to the reservoir 404 and/or as affected by the incoming flow of the aqueous fluid 408 at the juncture 406. Alternatively, the second fluid 410 may be subjected and/or directed to flow in or out of the reservoir 404. For example, the reservoir 404 can be a channel directing the second fluid 410 from upstream to downstream, transporting the generated droplets.

The channel structure 400 at or near the juncture 406 may have certain geometric features that at least partly determine the sizes of the droplets formed by the channel structure 400. The channel segment 402 can have a height, h₀ and width, w, at or near the juncture 406. By way of example, the channel segment 402 can comprise a rectangular cross-section that leads to a reservoir 404 having a wider cross-section (such as in width or diameter). Alternatively, the cross-section of the channel segment 402 can be other shapes, such as a circular shape, trapezoidal shape, polygonal shape, or any other shapes. The top and bottom walls of the reservoir 404 at or near the juncture 406 can be inclined at an expansion angle, a. The expansion angle, a, allows the tongue (portion of the aqueous fluid 408 leaving channel segment 402 at junction 406 and entering the reservoir 404 before droplet formation) to increase in depth and facilitate decrease in curvature of the intermediately formed droplet. Droplet size may decrease with increasing expansion angle. The resulting droplet radius, R_{d}, may be predicted by the following equation for the aforementioned geometric parameters of h₀, w, and a:

$${R}_{d} \approx 0.44 ( 1 + 2.2 \sqrt{tan α} \frac{w}{{h}_{0}} ) \frac{{h}_{0}}{\sqrt{tan α}}$$

By way of example, for a channel structure with w=21 μm, h=21 μm, and α=3°, the predicted droplet size is 121 μm. In another example, for a channel structure with w=25 h=25 μm, and α=5°, the predicted droplet size is 123 μm. In another example, for a channel structure with w=28 μm, h=28 μm, and α=7°, the predicted droplet size is 124 μm.

In some instances, the expansion angle, α, may be between a range of from about 0.5° to about 4°, from about 0.1° to about 10°, or from about 0° to about 90°. For example, the expansion angle can be at least about 0.01°, 0.1°, 0.2°, 0.3°, 0.4°, 0.5°, 0.6°, 0.7°, 0.8°, 0.9°, 1°, 2°, 3°, 4°, 5°, 6°, 7°, 8°, 9°, 10°, 15°, 20°, 25°, 30°, 35°, 40°, 45°, 50°, 55°, 60°, 65°, 70°, 75°, 80°, 85°, or higher. In some instances, the expansion angle can be at most about 89°, 88°, 87°, 86°, 85°, 84°, 83°, 82°, 81°, 80°, 75°, 70°, 65°, 60°, 55°, 50°, 45°, 40°, 35°, 30°, 25°, 20°, 15°, 10°, 9°, 8°, 7°, 6°, 5°, 4°, 3°, 2°, 1°, 0.1°, 0.01°, or less. In some instances, the width, w, can be between a range of from about 100 micrometers (μm) to about 500 μm. In some instances, the width, w, can be between a range of from about 10 μm to about 200 μm. Alternatively, the width can be less than about 10 μm. Alternatively, the width can be greater than about 500 μm. In some instances, the flow rate of the aqueous fluid 408 entering the junction 406 can be between about 0.04 microliters (μL)/minute (min) and about 40 μL/min. In some instances, the flow rate of the aqueous fluid 408 entering the junction 406 can be between about 0.01 microliters (μL)/minute (min) and about 100 μL/min. Alternatively, the flow rate of the aqueous fluid 408 entering the junction 406 can be less than about 0.01 μL/min. Alternatively, the flow rate of the aqueous fluid 408 entering the junction 406 can be greater than about 40 μL/min, such as 45 μL/min, 50 μL/min, 55 μL/min, 60 μL/min, 65 μL/min, 70 μL/min, 75 μL/min, 80 μL/min, 85 μL/min, 90 μL/min, 95 μL/min, 100 μL/min, 110 μL/min, 120 μL/min, 130 μL/min, 140 μL/min, 150 μL/min, or greater. At lower flow rates, such as flow rates of about less than or equal to 10 microliters/minute, the droplet radius may not be dependent on the flow rate of the aqueous fluid 408 entering the junction 406.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

The throughput of droplet generation can be increased by increasing the points of generation, such as increasing the number of junctions (e.g., junction 406) between aqueous fluid 408 channel segments (e.g., channel segment 402) and the reservoir 404. Alternatively or in addition, the throughput of droplet generation can be increased by increasing the flow rate of the aqueous fluid 408 in the channel segment 402.

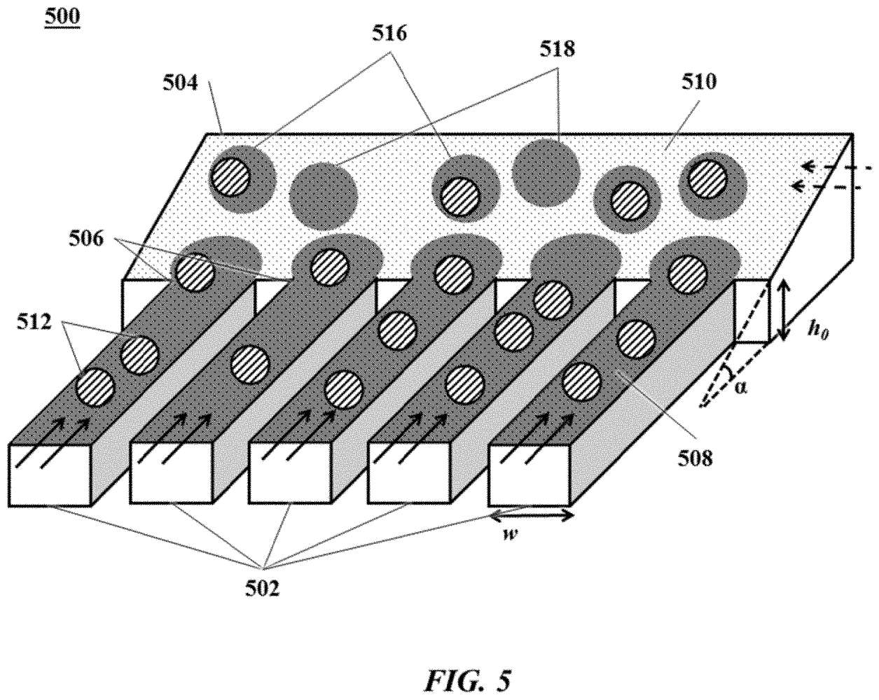

FIG. 5 shows an example of a microfluidic channel structure for increased droplet generation throughput. A microfluidic channel structure 500 can comprise a plurality of channel segments 502 and a reservoir 504. Each of the plurality of channel segments 502 may be in fluid communication with the reservoir 504. The channel structure 500 can comprise a plurality of channel junctions 506 between the plurality of channel segments 502 and the reservoir 504. Each channel junction can be a point of droplet generation. The channel segment 402 from the channel structure 400 in FIG. 4 and any description to the components thereof may correspond to a given channel segment of the plurality of channel segments 502 in channel structure 500 and any description to the corresponding components thereof. The reservoir 404 from the channel structure 400 and any description to the components thereof may correspond to the reservoir 504 from the channel structure 500 and any description to the corresponding components thereof.

Each channel segment of the plurality of channel segments 502 may comprise an aqueous fluid 508 that includes suspended beads 512. The reservoir 504 may comprise a second fluid 510 that is immiscible with the aqueous fluid 508. In some instances, the second fluid 510 may not be subjected to and/or directed to any flow in or out of the reservoir 504. For example, the second fluid 510 may be substantially stationary in the reservoir 504. In some instances, the second fluid 510 may be subjected to flow within the reservoir 504, but not in or out of the reservoir 504, such as via application of pressure to the reservoir 504 and/or as affected by the incoming flow of the aqueous fluid 508 at the junctures. Alternatively, the second fluid 510 may be subjected and/or directed to flow in or out of the reservoir 504. For example, the reservoir 504 can be a channel directing the second fluid 510 from upstream to downstream, transporting the generated droplets.

In operation, the aqueous fluid 508 that includes suspended beads 512 may be transported along the plurality of channel segments 502 into the plurality of junctions 506 to meet the second fluid 510 in the reservoir 504 to create droplets 516, 518. A droplet may form from each channel segment at each corresponding junction with the reservoir 504. At the juncture where the aqueous fluid 508 and the second fluid 510 meet, droplets can form based on factors such as the hydrodynamic forces at the juncture, flow rates of the two fluids 508, 510, fluid properties, and certain geometric parameters (e.g., w, h₀, α, etc.) of the channel structure 500, as described elsewhere herein. A plurality of droplets can be collected in the reservoir 504 by continuously injecting the aqueous fluid 508 from the plurality of channel segments 502 through the plurality of junctures 506. Throughput may significantly increase with the parallel channel configuration of channel structure 500. For example, a channel structure having five inlet channel segments comprising the aqueous fluid 508 may generate droplets five times as frequently than a channel structure having one inlet channel segment, provided that the fluid flow rate in the channel segments are substantially the same. The fluid flow rate in the different inlet channel segments may or may not be substantially the same. A channel structure may have as many parallel channel segments as is practical and allowed for the size of the reservoir. For example, the channel structure may have at least about 2, 3, 4, 5, 6, 7, 8, 9, 10, 20, 30, 40, 50, 60, 70, 80, 90, 100, 150, 500, 250, 300, 350, 400, 450, 500, 600, 700, 800, 900, 1000, 1500, 5000 or more parallel or substantially parallel channel segments.

The geometric parameters, w, h₀, and a, may or may not be uniform for each of the channel segments in the plurality of channel segments 502. For example, each channel segment may have the same or different widths at or near its respective channel junction with the reservoir 504. For example, each channel segment may have the same or different height at or near its respective channel junction with the reservoir 504. In another example, the reservoir 504 may have the same or different expansion angle at the different channel junctions with the plurality of channel segments 502. When the geometric parameters are uniform, beneficially, droplet size may also be controlled to be uniform even with the increased throughput. In some instances, when it is desirable to have a different distribution of droplet sizes, the geometric parameters for the plurality of channel segments 502 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size.

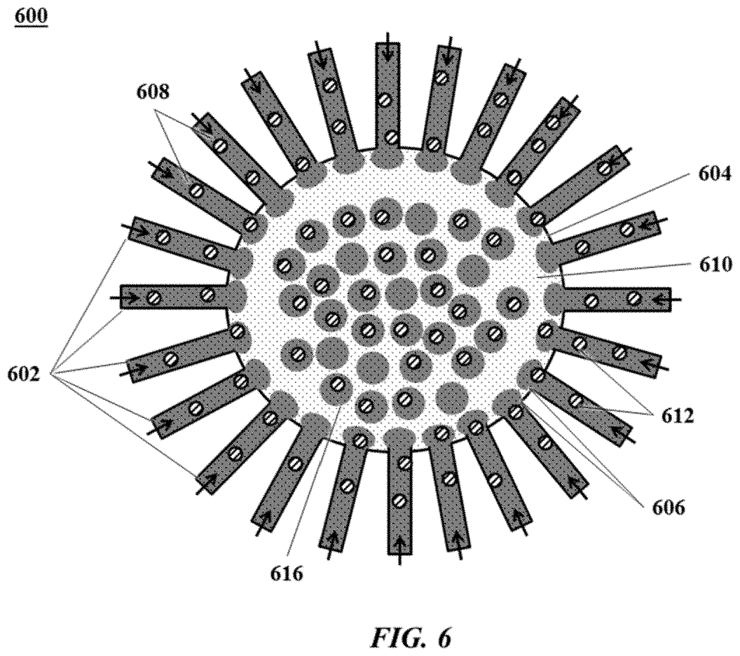

FIG. 6 shows another example of a microfluidic channel structure for increased droplet generation throughput. A microfluidic channel structure 600 can comprise a plurality of channel segments 602 arranged generally circularly around the perimeter of a reservoir 604. Each of the plurality of channel segments 602 may be in fluid communication with the reservoir 604. The channel structure 600 can comprise a plurality of channel junctions 606 between the plurality of channel segments 602 and the reservoir 604. Each channel junction can be a point of droplet generation. The channel segment 402 from the channel structure 400 in FIG. 2 and any description to the components thereof may correspond to a given channel segment of the plurality of channel segments 602 in channel structure 600 and any description to the corresponding components thereof. The reservoir 404 from the channel structure 400 and any description to the components thereof may correspond to the reservoir 604 from the channel structure 600 and any description to the corresponding components thereof.

Each channel segment of the plurality of channel segments 602 may comprise an aqueous fluid 608 that includes suspended beads 612. The reservoir 604 may comprise a second fluid 610 that is immiscible with the aqueous fluid 608. In some instances, the second fluid 610 may not be subjected to and/or directed to any flow in or out of the reservoir 604. For example, the second fluid 610 may be substantially stationary in the reservoir 604. In some instances, the second fluid 610 may be subjected to flow within the reservoir 604, but not in or out of the reservoir 604, such as via application of pressure to the reservoir 604 and/or as affected by the incoming flow of the aqueous fluid 608 at the junctures. Alternatively, the second fluid 610 may be subjected and/or directed to flow in or out of the reservoir 604. For example, the reservoir 604 can be a channel directing the second fluid 610 from upstream to downstream, transporting the generated droplets.

In operation, the aqueous fluid 608 that includes suspended beads 612 may be transported along the plurality of channel segments 602 into the plurality of junctions 606 to meet the second fluid 610 in the reservoir 604 to create a plurality of droplets 616. A droplet may form from each channel segment at each corresponding junction with the reservoir 604. At the juncture where the aqueous fluid 608 and the second fluid 610 meet, droplets can form based on factors such as the hydrodynamic forces at the juncture, flow rates of the two fluids 608, 610, fluid properties, and certain geometric parameters (e.g., widths and heights of the channel segments 602, expansion angle of the reservoir 604, etc.) of the channel structure 600, as described elsewhere herein. A plurality of droplets can be collected in the reservoir 604 by continuously injecting the aqueous fluid 608 from the plurality of channel segments 602 through the plurality of junctures 606. Throughput may significantly increase with the substantially parallel channel configuration of the channel structure 600. A channel structure may have as many substantially parallel channel segments as is practical and allowed for by the size of the reservoir. For example, the channel structure may have at least about 2, 3, 4, 5, 6, 7, 8, 9, 10, 20, 30, 40, 50, 60, 70, 80, 90, 100, 150, 200, 250, 300, 350, 400, 450, 500, 600, 700, 800, 900, 1000, 1500, 5000 or more parallel or substantially parallel channel segments. The plurality of channel segments may be substantially evenly spaced apart, for example, around an edge or perimeter of the reservoir. Alternatively, the spacing of the plurality of channel segments may be uneven.

The reservoir 604 may have an expansion angle, a (not shown in FIG. 6) at or near each channel juncture. Each channel segment of the plurality of channel segments 602 may have a width, w, and a height, h₀, at or near the channel juncture. The geometric parameters, w, h₀, and α, may or may not be uniform for each of the channel segments in the plurality of channel segments 602. For example, each channel segment may have the same or different widths at or near its respective channel junction with the reservoir 604. For example, each channel segment may have the same or different height at or near its respective channel junction with the reservoir 604.

The reservoir 604 may have the same or different expansion angle at the different channel junctions with the plurality of channel segments 602. For example, a circular reservoir (as shown in FIG. 6) may have a conical, dome-like, or hemispherical ceiling (e.g., top wall) to provide the same or substantially same expansion angle for each channel segments 602 at or near the plurality of channel junctions 606. When the geometric parameters are uniform, beneficially, resulting droplet size may be controlled to be uniform even with the increased throughput. In some instances, when it is desirable to have a different distribution of droplet sizes, the geometric parameters for the plurality of channel segments 602 may be varied accordingly.

In some instances, at least about 50% of the droplets generated can have uniform size. In some instances, at least about 55%, 60%, 65%, 70%, 75%, 80%, 85%, 90%, 95%, 96%, 97%, 98%, 99%, or greater of the droplets generated can have uniform size. Alternatively, less than about 50% of the droplets generated can have uniform size. The beads and/or biological particle injected into the droplets may or may not have uniform size.

The channel networks, e.g., as described above or elsewhere herein, can be fluidly coupled to appropriate fluidic components. For example, the inlet channel segments are fluidly coupled to appropriate sources of the materials they are to deliver to a channel junction. These sources may include any of a variety of different fluidic components, from simple reservoirs defined in or connected to a body structure of a microfluidic device, to fluid conduits that deliver fluids from off-device sources, manifolds, fluid flow units (e.g., actuators, pumps, compressors) or the like. Likewise, the outlet channel segment (e.g., channel segment 208, reservoir 604, etc.) may be fluidly coupled to a receiving vessel or conduit for the partitioned cells for subsequent processing. Again, this may be a reservoir defined in the body of a microfluidic device, or it may be a fluidic conduit for delivering the partitioned cells to a subsequent process operation, instrument or component.

The methods and systems described herein may be used to greatly increase the efficiency of single cell applications and/or other applications receiving droplet-based input. For example, following the sorting of occupied cells and/or appropriately-sized cells, subsequent operations that can be performed can include generation of amplification products, purification (e.g., via solid phase reversible immobilization (SPRI)), further processing (e.g., shearing, ligation of functional sequences, and subsequent amplification (e.g., via PCR)). These operations may occur in bulk (e.g., outside the partition). In the case where a partition is a droplet in an emulsion, the emulsion can be broken and the contents of the droplet pooled for additional operations. Additional reagents that may be co-partitioned along with the barcode bearing bead may include oligonucleotides to block ribosomal RNA (rRNA) and nucleases to digest genomic DNA from cells. Alternatively, rRNA removal agents may be applied during additional processing operations. The configuration of the constructs generated by such a method can help minimize (or avoid) sequencing of the poly-T sequence during sequencing and/or sequence the 5′ end of a polynucleotide sequence. The amplification products, for example, first amplification products and/or second amplification products, may be subject to sequencing for sequence analysis. In some cases, amplification may be performed using the Partial Hairpin Amplification for Sequencing (PHASE) method.

A variety of applications require the evaluation of the presence and quantification of different biological particle or organism types within a population of biological particles, including, for example, microbiome analysis and characterization, environmental testing, food safety testing, epidemiological analysis, e.g., in tracing contamination or the like.

### Computer Systems

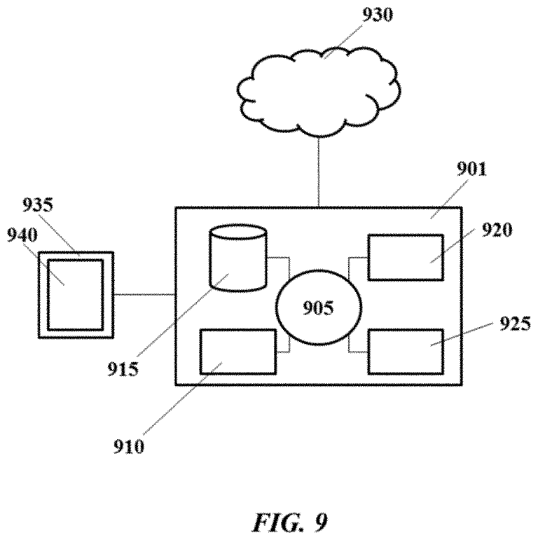

The present disclosure provides computer systems that are programmed to implement methods of the disclosure. FIG. 9 shows a computer system 901 that is programmed or otherwise configured to, e.g., control a microfluidics system (e.g., fluid flow), sort occupied droplets from unoccupied droplets, polymerize a polymerizable material (e.g., by delivering a stimulus), perform sequencing applications, or generate and maintain a library of particles including multiple gel components. The computer system 901 can regulate various aspects of the present disclosure, such as, for example, a fluid flow rate in one or more channels in a microfluidic structure and polymerization application units. The computer system 901 can be an electronic device of a user or a computer system that is remotely located with respect to the electronic device. The electronic device can be a mobile electronic device.

The computer system 901 includes a central processing unit (CPU, also “processor” and “computer processor” herein) 905, which can be a single core or multi core processor, or a plurality of processors for parallel processing. The computer system 901 also includes memory or memory location 910 (e.g., random-access memory, read-only memory, flash memory), electronic storage unit 915 (e.g., hard disk), communication interface 920 (e.g., network adapter) for communicating with one or more other systems, and peripheral devices 925, such as cache, other memory, data storage and/or electronic display adapters. The memory 910, storage unit 915, interface 920 and peripheral devices 925 are in communication with the CPU 905 through a communication bus (solid lines), such as a motherboard. The storage unit 915 can be a data storage unit (or data repository) for storing data. The computer system 901 can be operatively coupled to a computer network (“network”) 930 with the aid of the communication interface 920. The network 930 can be the Internet, an internet and/or extranet, or an intranet and/or extranet that is in communication with the Internet. The network 930 in some cases is a telecommunication and/or data network. The network 930 can include one or more computer servers, which can enable distributed computing, such as cloud computing. The network 930, in some cases with the aid of the computer system 901, can implement a peer-to-peer network, which may enable devices coupled to the computer system 901 to behave as a client or a server.

The CPU 905 can execute a sequence of machine-readable instructions, which can be embodied in a program or software. The instructions may be stored in a memory location, such as the memory 910. The instructions can be directed to the CPU 905, which can subsequently program or otherwise configure the CPU 905 to implement methods of the present disclosure. Examples of operations performed by the CPU 905 can include fetch, decode, execute, and writeback.

The CPU 905 can be part of a circuit, such as an integrated circuit. One or more other components of the system 901 can be included in the circuit. In some cases, the circuit is an application specific integrated circuit (ASIC).

The storage unit 915 can store files, such as drivers, libraries and saved programs. The storage unit 915 can store user data, e.g., user preferences and user programs. The computer system 901 in some cases can include one or more additional data storage units that are external to the computer system 901, such as located on a remote server that is in communication with the computer system 901 through an intranet or the Internet.

The computer system 901 can communicate with one or more remote computer systems through the network 930. For instance, the computer system 901 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 901 via the network 930.

Methods as described herein can be implemented by way of machine (e.g., computer processor) executable code stored on an electronic storage location of the computer system 901, such as, for example, on the memory 910 or electronic storage unit 915. The machine executable or machine readable code can be provided in the form of software. During use, the code can be executed by the processor 905. In some cases, the code can be retrieved from the storage unit 915 and stored on the memory 910 for ready access by the processor 905. In some situations, the electronic storage unit 915 can be precluded, and machine-executable instructions are stored on memory 910.

The code can be pre-compiled and configured for use with a machine having a processor adapted to execute the code, or can be compiled during runtime. The code can be supplied in a programming language that can be selected to enable the code to execute in a pre-compiled or as-compiled fashion.

Aspects of the systems and methods provided herein, such as the computer system 901, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 901 can include or be in communication with an electronic display 935 that comprises a user interface (UI) 940 for providing, for example, results of a sequencing analysis. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 905. The algorithm can, for example, perform sequencing, etc.

Devices, systems, compositions and methods of the present disclosure may be used for various applications, such as, for example, processing a single analyte (e.g., RNA, DNA, or protein) or multiple analytes (e.g., DNA and RNA, DNA and protein, RNA and protein, or RNA, DNA and protein) form a single cell. For example, a biological particle (e.g., a cell or cell bead) is partitioned in a partition (e.g., droplet), and multiple analytes from the biological particle are processed for subsequent processing. The multiple analytes may be from the single cell. This may enable, for example, simultaneous proteomic, transcriptomic and genomic analysis of the cell.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-77. (canceled)

78. A particle for use in processing or analyzing an analyte from a sample, comprising:
a first gel comprising said analyte; and
a second gel separate from but in direct physical contact with said first gel,
wherein said second gel at least partially encompasses said first gel, or said first gel at least partially encompasses said second gel.

79. The particle of claim 78, wherein said first gel is comprised of a first material and said second gel is comprised of a second material different than said first material.

80. The particle of claim 79, wherein said particle further comprises a third gel separate from said first gel and said second gel, and wherein said third gel is comprised of a third material different than said first material.

81. The particle of claim 78, wherein said analyte is a nucleic acid molecule.

82. The particle of claim 78, wherein said analyte is included within a cell.

83. The particle of claim 78, wherein said second gel comprises a reagent for processing or analyzing said analyte.

84. The particle of claim 78, wherein said first gel further comprises a reagent for processing or analyzing said analyte.

85. The particle of claim 78, wherein said second gel substantially encompasses said first gel.

86. The particle of claim 78, wherein said first gel substantially encompasses said second gel.

87. The particle of claim 78, wherein said first gel and said second gel comprise a same material.

88. The particle of claim 78, wherein said first gel or said second gel comprises a reagent for processing or analyzing said analyte, wherein said reagent is selected from the group consisting of temperature-sensitive enzymes, pH-sensitive enzymes, light-sensitive enzymes, reverse transcriptase, proteases, ligase, polymerases, restriction enzymes, nucleases, protease inhibitors, and nuclease inhibitors.

89. The particle of claim 78, wherein said first gel or said second gel comprises a reagent for processing or analyzing said analyte, wherein said reagent is selected from the group consisting of enzymes, fluorophores, oligonucleotides, primers, nucleic acid barcode molecules, barcodes, buffers, deoxynucleotide triphosphates, detergents, reducing agents, chelating agents, oxidizing agents, nanoparticles, and antibodies.

90. The particle of claim 78, wherein said particle comprises a plurality of nucleic acid barcode molecules coupled thereto.

91. The particle of claim 78, wherein said particle further comprises a third gel separate from said first gel and said second gel.

92. A method of forming a particle for use in processing or analyzing an analyte from a sample, comprising:
(a) providing a first gel comprising said analyte;
(b) generating a droplet comprising said first gel, and a polymerizable material; and
(c) subjecting said polymerizable material to conditions sufficient to generate a second gel separate from said first gel,
wherein said second gel at least partially encompasses said first gel, and wherein said first gel and said second gel are disruptable or dissolvable upon application of a stimulus.

93. The method of claim 92, wherein said first gel further comprises a second analyte.

94. The method of claim 92, wherein said first gel further comprises a reagent for processing or analyzing said analyte.

95. The method of claim 92, wherein said droplet comprises a second analyte.

96. The method of claim 95, wherein said second gel comprises said second analyte.

97. The method of claim 92, wherein said stimulus is selected from the group consisting of chemical triggers, bulk changes, biological triggers, light triggers, thermal triggers, magnetic triggers, and any combination thereof.
